(19) United States
(12) Patent Application Publication
(10) Pub. No.: US 2024/0197636 A1
(43) Pub. Date: Jun. 20, 2024

(54) TISSUE-SPECIFIC NUCLEIC ACID DELIVERY BY MIXED CATIONIC LIPID PARTICLES

(71) Applicant: OMEGA THERAPEUTICS, INC., Cambridge, MA (US)

(72) Inventors: Vishwesh Ashok Patil, Arlington, MA (US); Can Sarisozen, Westford, MA (US); Daniel Ferreira Gomes Costa, Cambridge, MA (US)

(73) Assignee: OMEGA THERAPEUTICS, INC., Cambridge, MA (US)

(21) Appl. No.: 18/556,563

(22) PCT Filed: Apr. 21, 2022
(86) PCT No.: PCT/US2022/025674

§ 371 Date: Oct. 20, 2023

Related U.S. Application Data
(60) Provisional application No. 63/178,050, filed on Apr. 22, 2021.

(51) Int. Cl. A61K 9/16; A61K 47/54; A61P 1/16; C12N 15/113

(52) U.S. Cl. CPC A61K 9/1617; A61K 47/543; A61P 1/16; C12N 15/113; C12N 2310/14

(57) ABSTRACT

The instant disclosure relates to nucleic acid-lipid particles that mix ionizable and cationic lipids, which preferentially localize and deliver associated cargoes to the liver when administered parenterally, and which optionally can be used to deliver associated cargoes to tissues to which such particles are directly injected. The instant disclosure provides compositions comprising such lipid particles, optionally in association with a therapeutic agent (e.g., a therapeutic mRNA and/or nucleic acid controller system), as well as methods and kits for delivering a lipid particle-associated therapeutic agent and/or treating a disease or disorder, e.g., a liver disease or disorder, in a subject, using the lipid particle compositions provided herein.

### CROSS-REFERENCE TO RELATED APPLICATION

The present application is related to and claims priority under 35 U.S.C. § 119(e) to U.S. provisional patent application No. 63/178,050, entitled “Tissue-Specific Nucleic Acid Delivery by Mixed Cationic Lipid Particles,” filed Apr. 22, 2021. The entire content of the aforementioned patent application is incorporated herein by this reference.

### FIELD OF THE INVENTION

The current disclosure relates to lipid-based compositions and methods useful in administering nucleic acid-based therapies. In particular, the disclosure relates to mixed cationic/ionizable lipid particles for delivery of nucleic acids to a subject, including for treatment or prevention of diseases and disorders of a subject, including diseases or disorders of or involving liver tissues of a subject.

### BACKGROUND OF THE INVENTION

Liver disease accounts for approximately 2 million deaths per year worldwide, which includes approximately more than 1 million deaths due to the complications of cirrhosis, viral hepatitis and hepatocellular carcinoma (HCC). Liver cirrhosis and liver cancer account for almost 3.5% of all deaths worldwide. Liver transplantation is the second most prevalent solid organ transplantation, used as a last resort treatment option to address many liver conditions, and unfortunately less than 10% of global transplantation needs are currently met. Although some treatments exist for liver diseases and disorders that can help delay or even prevent the need for a liver transplantation, such known treatments are by no means completely restorative, and a major challenge in the field remains to develop therapeutic agents that effectively treat liver diseases without prohibitively harming a treated subject.

Nucleic acid therapies offer tremendous potential for treatment of diseases at the level of individual, targeted genes. However, safe and effective delivery systems are essential for realizing the full promise of nucleic acid therapeutics. Non-specific delivery of nucleic acid therapeutics to all organs and tissues can often result in off-site (non-targeted and/or off-target) effects and toxicity. Delivery of nucleic acid therapeutics preferentially to an organ or tissue of interest in which a specific action is desirable is a continuing goal for drug delivery and delivery of nucleic acid-based agents in particular. The concept of only targeting the cause of a disease without harming other parts of the body was described by Ehrlich 120 years ago. However, there are still effectively no options for nanoparticle delivery systems that are capable of targeting specific tissues without introducing ligand-based targeting strategies (with the latter also referred to as “active targeting”). There is therefore a previously unmet need in the art for vehicles that are capable of achieving organ-specific delivery of nucleic acid cargoes based only upon the structural components of such formulations (rather than by ligand-based active targeting strategies). In particular, because the liver is a key target organ for gene therapy, there is also a specific need in the art for such vehicles capable of selectively delivering nucleic acid cargoes to the liver.

### BRIEF SUMMARY OF THE INVENTION

The instant disclosure is based, at least in part, upon identification of lipid-based nanoparticle compositions and formulations capable of specifically targeting a cargo moiety (e.g., a nucleic acid cargo) to the liver and liver tissues of a subject, without requiring a ligand-based targeting strategy. In particular, the instant disclosure relates to the surprising discovery that introduction of a second ionizable or cationic lipid to nucleic acid-lipid particle formulations containing the cationic lipid DOTAP (1,2-dioleoyl-3-trimethylammonium-propane) could effectively shift such particle formulations to be liver-specific in their delivery of nucleic acid cargoes, and robustly achieve delivery of even large nucleic acid modulatory controller cargoes to nuclei of liver cells of a particle-treated subject. Inclusion of the ionizable lipid C12-200 (1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol)) within DOTAP-containing particles (thereby forming mixed cationic lipid particles) was specifically discovered herein to shift the tropism of vectors specifically to liver tissues without requiring a further active-targeting component in the LNPs, with improvements noted even as compared to MC3-containing lipid particles (which have been previously described in the art to achieve selective delivery of nucleic acid cargoes to the liver). The instant disclosure therefore provides lipid particles that are capable of effective delivery of nucleic acid cargoes selectively to the liver upon systemic administration (e.g., via intravenous (IV) injection), with such nucleic acid cargoes including, e.g., nucleic acid modulating controllers, therapeutic mRNAs, as well as RNA interference (RNAi) and antisense agents (siRNAs, miRNAs, etc.), among others.

In one aspect, the instant disclosure provides a nucleic acid-lipid particle for delivering a nucleic acid cargo to a liver tissue of a subject, the nucleic acid-lipid particle including 1,2-dioleoyl-3-trimethylammonium-propane (DOTAP) at about 10 mol % to about 50 mol % of the total lipid present in the nucleic acid-lipid particle; and ionizable lipid at about 5 mol % to about 50 mol % of the total lipid present in the nucleic acid-lipid particle.

In one embodiment, the ionizable lipid is 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), N4-Cholesteryl-Spermine HCl (GL67), N1-[2-((1S)-1-[(3-aminopropyl)amino]-4-[di(3-amino-propyl)amino]butylcarboxamido)ethyl]-3,4-di[oleyloxy]-benzamide (MVL5) and/or polymeric branched polyethyleneimine (bPEI).

In certain embodiments, the particle includes one or more non-cationic lipids present at about 25 mol % to about 85 mol % of the total lipid present in the lipid-nucleic acid particle. Optionally, the one or more non-cationic lipids include a structural lipid, e.g., cholesterol, (3-sitosterol or one or more derivatives thereof.

In embodiments, the particle includes cholesterol, β-sitosterol or one or more derivatives thereof present at about 10 mol % to about 75 mol % of the total lipid present in the nucleic acid-lipid particle. Optionally, the particle includes cholesterol, β-sitosterol or one or more derivatives thereof present at about 20 mol % to about 65 mol % of the total lipid present in the nucleic acid-lipid particle. Optionally, the particle includes cholesterol, β-sitosterol or one or more derivatives thereof present at about 24 mol % to about 64 mol % of the total lipid present in the nucleic acid-lipid particle. Optionally, the particle includes cholesterol, β-sitosterol or one or more derivatives thereof at about 24 mol % of the total lipid present in the nucleic acid-lipid particle, about 34 mol % of the total lipid present in the nucleic acid-lipid particle, about 38 mol % of the total lipid present in the nucleic acid-lipid particle, about 44 mol % of the total lipid present in the nucleic acid-lipid particle, about 54 mol % of the total lipid present in the nucleic acid-lipid particle or about 64 mol % of the total lipid present in the nucleic acid-lipid particle.

In one embodiment, the particle includes one or more non-cationic lipid other than cholesterol, β-sitosterol or a derivative thereof. Optionally, the one or more non-cationic lipid other than cholesterol, β-sitosterol or derivatives thereof is present at about 5 mol % to about 50 mol % of the total lipid present in the lipid-nucleic acid particle. Optionally, the one or more non-cationic lipid other than cholesterol, β-sitosterol or derivatives thereof is present at about 5 mol % to about 30 mol % of the total lipid present in the lipid-nucleic acid particle. Optionally, the one or more non-cationic lipid other than cholesterol, β-sitosterol or derivatives thereof is present at about 5 mol % to about 10 mol % of the total lipid present in the lipid-nucleic acid particle.

In certain embodiments, the particle includes one or more non-cationic lipid other than cholesterol, β-sitosterol or derivatives thereof at a level of about 6 mol % of the total lipid present in the nucleic acid-lipid particle, about 7 mol % of the total lipid present in the nucleic acid-lipid particle, about 7.5 mol % of the total lipid present in the nucleic acid-lipid particle, about 8 mol % of the total lipid present in the nucleic acid-lipid particle, about 9 mol % of the total lipid present in the nucleic acid-lipid particle, about 10 mol % of the total lipid present in the nucleic acid-lipid particle, about 16 mol % of the total lipid present in the nucleic acid-lipid particle, about 26 mol % of the total lipid present in the nucleic acid-lipid particle, about 36 mol % of the total lipid present in the nucleic acid-lipid particle, or about 46 mol % of the total lipid present in the nucleic acid-lipid particle.

In embodiments, the one or more non-cationic lipid other than cholesterol, β-sitosterol or derivatives thereof is 1,2-Dioleoyl-sn-glycero-3-phosphoethanolamine (DOPE), 1,2-dioleoyl-sn-glycero-3-phosphocholine (DOPC) and/or 1,2-Distearoyl-sn-glycero-3-phosphocholine (DSPC). Optionally, the one or more non-cationic lipid other than cholesterol, β-sitosterol or derivatives thereof is DOPE.

In one embodiment, the nucleic acid-lipid particle does not include a PEG-lipid conjugate. Optionally, the nucleic acid-lipid particle does not include PEG.

In certain embodiments, the nucleic acid-lipid particle is a component of a multi-dose therapy.

In an embodiment, the particle includes a conjugated lipid that inhibits aggregation of particles present at 0.01 to 3% of the total lipid present. Optionally, the conjugated lipid is or includes a polyethyleneglycol (PEG)-lipid conjugate. Optionally, the PEG of the PEG-lipid conjugate has an average molecular weight of from 550 daltons to 3000 daltons. Optionally, the PEG-lipid conjugate is a PEG2000-lipid conjugate. Optionally, the PEG2000-lipid conjugate is or includes one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k). Optionally, the PEG2000-lipid conjugate is 1,2-Dimyristoyl-rac-glycero-3-methoxypolyethylene glycol-2000 (DMG-PEG2k). Optionally, the nucleic acid-lipid particle includes a PEG-lipid conjugate at a level of about 0.5 mol % of the total lipid present in the nucleic acid-lipid particle, about 1.0 mol % of the total lipid present in the nucleic acid-lipid particle, about 1.5 mol % of the total lipid present in the nucleic acid-lipid particle, or about 2.0 mol % of the total lipid present in the nucleic acid-lipid particle.

In one embodiment, the nucleic acid cargo is or comprises a synthetic or naturally occurring RNA or DNA, or derivatives thereof. Optionally, the nucleic acid cargo is a modified RNA. Optionally, the modified RNA is a modified mRNA, a modified antisense oligonucleotide and/or a modified siRNA. Optionally, the modified mRNA encodes a nucleic acid modulating controller.

In embodiments, the nucleic acid cargo includes one or more of the following modifications: 2′-O-methyl modified nucleotides, a nucleotide including a 5′-phosphorothioate group, a terminal nucleotide linked to a cholesteryl derivative, a 2′-deoxy-2′-fluoro modified nucleotide, a 5′-methoxy-modified nucleotide (e.g., 5′-methoxyuridine), a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base including nucleotide; internucleoside linkages or backbones including phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

In certain embodiments, the liver tissue is or includes hepatocytes, vascular cells (i.e. hepatic sinusoids), hepatic stellate cells, endothelial cells, fibroblasts, mesenchymal cells, immune cells, cancer cells, Kupffer cells, astrocytes, oval-shaped vascular endothelial cells, liver-derived stem/progenitor cells, and/or stem/progenitor or cancer cells derived from non-liver tissue.

In one embodiment, the particle includes DOTAP at a level of about 10 mol % of the total lipid present in the nucleic acid-lipid particle, about 20 mol % of the total lipid present in the nucleic acid-lipid particle, about 30 mol % of the total lipid present in the nucleic acid-lipid particle, about 40 mol % of the total lipid present in the nucleic acid-lipid particle, or about 50 mol % of the total lipid present in the nucleic acid-lipid particle.

Another aspect of the instant disclosure provides a nucleic acid-lipid particle for delivering a nucleic acid cargo to a liver tissue of a subject, the nucleic acid-lipid particle including DOTAP at about 50 mol % of the total lipid present in the nucleic acid-lipid particle and ionizable lipid present at about 18 mol % of the total lipid present in the nucleic acid-lipid particle.

In one embodiment, the ionizable lipid is 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200).

In certain embodiments, the particle further includes cholesterol, β-sitosterol or derivatives thereof at about 24 mol % of the total lipid present in the nucleic acid-lipid particle. Optionally, the particle further includes one or more non-cationic lipid other than cholesterol, β-sitosterol or derivatives thereof at about 6 mol % of the total lipid present in the nucleic acid-lipid particle. Optionally, the one or more non-cationic lipid other than cholesterol, β-sitosterol or derivatives thereof is DOPE. Optionally, the particle further includes a polyethyleneglycol (PEG)-lipid conjugate. Optionally, the PEG-lipid conjugate includes about 2.0 mol % of the total lipid present in the nucleic acid-lipid particle. Optionally, the PEG-lipid conjugate is 1,2-dimyristoyl-rac-glycero-3-methoxypolyethylene glycol-2000 (DMG-PEG2k).

An additional aspect of the instant disclosure provides a nucleic acid-lipid particle for delivering a nucleic acid cargo to a liver tissue of a subject, the nucleic acid-lipid particle including DOTAP at about 40 mol % of the total lipid present in the nucleic acid-lipid particle and ionizable lipid present at about 18 mol % of the total lipid present in the nucleic acid-lipid particle.

In certain embodiments, the particle further includes one or more non-cationic lipid other than cholesterol, β-sitosterol or derivatives thereof at about 16 mol % of the total lipid present in the nucleic acid-lipid particle.

One aspect of the instant disclosure provides a nucleic acid-lipid particle for delivering a nucleic acid cargo to a liver tissue of a subject, the nucleic acid-lipid particle including DOTAP at about 30 mol % of the total lipid present in the nucleic acid-lipid particle and ionizable lipid present at about 18 mol % of the total lipid present in the nucleic acid-lipid particle.

In one embodiment, the particle further includes one or more non-cationic lipid other than cholesterol, β-sitosterol or derivatives thereof at about 26 mol % of the total lipid present in the nucleic acid-lipid particle.

Another aspect of the instant disclosure provides a nucleic acid-lipid particle for delivering a nucleic acid cargo to a liver tissue of a subject, the nucleic acid-lipid particle including DOTAP at about 20 mol % of the total lipid present in the nucleic acid-lipid particle and ionizable lipid present at about 18 mol % of the total lipid present in the nucleic acid-lipid particle.

In one embodiment, the particle further includes one or more non-cationic lipid other than cholesterol, β-sitosterol or derivatives thereof at about 36 mol % of the total lipid present in the nucleic acid-lipid particle.

A further aspect of the instant disclosure provides a nucleic acid-lipid particle for delivering a nucleic acid cargo to a liver tissue of a subject, the nucleic acid-lipid particle including DOTAP at about 10 mol % of the total lipid present in the nucleic acid-lipid particle and ionizable lipid present at about 18 mol % of the total lipid present in the nucleic acid-lipid particle.

In one embodiment, the particle further includes one or more non-cationic lipid other than cholesterol, β-sitosterol or derivatives thereof present at about 46 mol % of the total lipid present in the nucleic acid-lipid particle.

An additional aspect of the instant disclosure provides a nucleic acid-lipid particle for delivering a nucleic acid cargo to a liver tissue of a subject, the nucleic acid-lipid particle including DOTAP at about 50 mol % of the total lipid present in the nucleic acid-lipid particle; ionizable lipid present at about 18 mol % of the total lipid present in the nucleic acid-lipid particle; and one or more non-cationic lipid other than cholesterol, β-sitosterol or derivatives thereof present at about 7.0 mol % of the total lipid present in the nucleic acid-lipid particle.

In one embodiment, the particle further includes a PEG-lipid conjugate at about 1.0 mol % of the total lipid present in the nucleic acid-lipid particle.

Another aspect of the instant disclosure provides a nucleic acid-lipid particle for delivering a nucleic acid cargo to a liver tissue of a subject, the nucleic acid-lipid particle including DOTAP at about 50 mol % of the total lipid present in the nucleic acid-lipid particle; ionizable lipid present at about 18 mol % of the total lipid present in the nucleic acid-lipid particle; and one or more non-cationic lipid other than cholesterol, β-sitosterol or derivatives thereof present at about 7.5 mol % of the total lipid present in the nucleic acid-lipid particle.

In one embodiment, the particle further includes a PEG-lipid conjugate at about 0.5 mol % of the total lipid present in the nucleic acid-lipid particle.

A further aspect of the instant disclosure provides a nucleic acid-lipid particle for delivering a nucleic acid cargo to a liver tissue of a subject, the nucleic acid-lipid particle including DOTAP at about 50 mol % of the total lipid present in the nucleic acid-lipid particle; ionizable lipid present at about 18 mol % of the total lipid present in the nucleic acid-lipid particle; and one or more non-cationic lipid other than cholesterol, β-sitosterol or derivatives thereof present at about 8.0 mol % of the total lipid present in the nucleic acid-lipid particle.

In one embodiment, the particle does not include a PEG-lipid conjugate. Optionally, the nucleic acid-lipid particle does not include PEG.

An additional aspect of the instant disclosure provides a nucleic acid-lipid particle for delivering a nucleic acid cargo to a liver tissue of a subject, the nucleic acid-lipid particle including DOTAP at about 40 mol % of the total lipid present in the nucleic acid-lipid particle; ionizable lipid present at about 18 mol % of the total lipid present in the nucleic acid-lipid particle; and cholesterol, β-sitosterol or derivatives thereof present at about 34 mol % of the total lipid present in the nucleic acid-lipid particle.

In certain embodiments, the nucleic acid-lipid particle further includes one or more non-cationic lipid other than cholesterol, β-sitosterol or derivatives thereof present at about 6.0 mol % of the total lipid present in the nucleic acid-lipid particle.

Another aspect of the instant disclosure provides a nucleic acid-lipid particle for delivering a nucleic acid cargo to a liver tissue of a subject, the nucleic acid-lipid particle including DOTAP at about 30 mol % of the total lipid present in the nucleic acid-lipid particle; ionizable lipid present at about 18 mol % of the total lipid present in the nucleic acid-lipid particle; and cholesterol, 3-sitosterol or derivatives thereof present at about 44 mol % of the total lipid present in the nucleic acid-lipid particle.

A further aspect of the instant disclosure provides a nucleic acid-lipid particle for delivering a nucleic acid cargo to a liver tissue of a subject, the nucleic acid-lipid particle including DOTAP at about 20 mol % of the total lipid present in the nucleic acid-lipid particle; ionizable lipid present at about 18 mol % of the total lipid present in the nucleic acid-lipid particle; and cholesterol, 3-sitosterol or derivatives thereof present at about 54 mol % of the total lipid present in the nucleic acid-lipid particle.

An additional aspect of the instant disclosure provides a nucleic acid-lipid particle for delivering a nucleic acid cargo to a liver tissue of a subject, the nucleic acid-lipid particle including DOTAP at about 10 mol % of the total lipid present in the nucleic acid-lipid particle; ionizable lipid present at about 18 mol % of the total lipid present in the nucleic acid-lipid particle; and cholesterol, β-sitosterol or derivatives thereof present at about 64 mol % of the total lipid present in the nucleic acid-lipid particle.

In another aspect, the instant disclosure provides a pharmaceutical composition that includes a nucleic acid-lipid particle of the instant disclosure.

In certain embodiments, the pharmaceutical composition is formulated for parenteral administration. Optionally, the pharmaceutical composition is formulated for intravenous injection.

In embodiments, the pharmaceutical composition is formulated for direct injection into the liver tissue.

In one embodiment, the nucleic acid-lipid particle or pharmaceutical composition of the instant disclosure is administered to treat a liver disease or disorder in a subject in need thereof. Optionally, the liver disease or disorder is biliary atresia, Alagille Syndrome, alpha-1 antitrypsin deficiency, Tyrosinemia, neonatal hepatitis, hepatitis C virus infection, hepatitis B virus infection, hepatitis A virus infection, hepatocellular carcinoma, and/or Wilson's disease.

Another aspect of the instant disclosure provides an injectate that includes the nucleic acid-lipid particle or pharmaceutical composition of the instant disclosure.

An additional aspect of the instant disclosure provides a method for delivering a nucleic acid cargo to a liver tissue of a subject that includes administering the nucleic acid-lipid particle, pharmaceutical composition, or injectate of the instant disclosure to the subject.

A further aspect of the instant disclosure provides a method for treating or preventing a disease or disorder in a subject, the method including administering the nucleic acid-lipid particle, pharmaceutical composition, or injectate of the instant disclosure the subject.

In embodiments, the nucleic acid-lipid particle, pharmaceutical composition or injectate is administered intravenously and expression of the nucleic acid cargo in cells of the liver tissue of the subject occurs at a level that is at least two-fold higher than expression of the nucleic acid cargo in cells of lung, heart, spleen, ovary, pancreas, kidney and/or other organ or tissue of the subject. Optionally, expression of the nucleic acid cargo in cells of the liver tissue of the subject is at least three-fold higher, optionally at least four-fold higher, optionally at least five-fold higher, optionally at least six-fold higher, optionally at least seven-fold higher, optionally at least eight-fold higher, optionally at least nine-fold higher, optionally at least ten-fold higher, optionally at least eleven-fold higher, optionally at least twelve-fold higher, optionally at least thirteen-fold higher, optionally at least fourteen-fold higher, optionally at least fifteen-fold higher, optionally at least twenty-fold higher, than expression of the nucleic acid cargo in cells of lung, heart, spleen, ovary, pancreas and/or kidney of the subject.

In some embodiments, the nucleic acid-lipid particle, pharmaceutical composition, or injectate of the instant disclosure is administered intravenously, and the nucleic acid-lipid particle localizes to the liver tissue of the subject at an at least two-fold higher concentration than the concentration of the nucleic acid-lipid particle in one or more of the following other tissues of the subject: lung, heart, spleen, ovaries and pancreas. Optionally, an at least three-fold, at least four-fold, at least five-fold, or an at least six-fold higher concentration of the nucleic acid-lipid particle is present in liver as compared to the concentration of the nucleic acid-lipid particle in one or more of the following other tissues of the subject: lung, heart, spleen, ovaries and/or pancreas.

In certain embodiments, administration of the nucleic acid-lipid particle, pharmaceutical composition, or injectate is performed to treat or prevent one or more of the following: a liver disease or disorder (e.g., biliary atresia, Alagille Syndrome, alpha-1 antitrypsin deficiency, Tyrosinemia, neonatal hepatitis, hepatitis C virus infection, hepatitis B virus infection, hepatitis A virus infection, hepatocellular carcinoma, and/or Wilson's disease); a joint disease or disorder (e.g., rheumatoid arthritis, psoriatic arthritis, gout, tendinitis, bursitis, Carpal Tunnel Syndrome and/or osteoarthritis); an inflammatory disease or disorder (e.g., inflammatory bowel disease, peritonitis, osteomyelitis, cachexia, pancreatitis, trauma induced shock, bronchial asthma, allergic rhinitis, cystic fibrosis, acute bronchitis, acute intense bronchitis, osteoarthritis, rheumatoid arthritis, infectious arthritis, post-infectious arthritis, gonocoele arthritis, tuberculous arthritis, arthritis, osteoarthritis, gout, spondyloarthropathies, ankylosing spondylitis, arthritis associated with vasculitis syndrome, nodular polyarteritis nervosa, irritable vasculitis, rugenic granulomatosis, rheumatoid polyposis myalgia, arthritis cell arteritis, calcium polycystic arthropathy, caustic gout, non-arthritic rheumatism, bursitis, hay fever, suppurative inflammation (e.g., tennis elbow), neuropathic joint disease, hemarthrosic, Henoch-Schlein purpura, hypertrophic osteoarthritis, multisized hemorrhoids, scoliosis, hemochromatosis, hyperlipoproteinemia, hypogammaglobulinemia, COPD, acute respiratory distress syndrome, acute lung injury, broncho-pulmonary dysplasia and/or systemic lupus erythematosus (SLE)); and an epidermal disease or disorder (e.g., psoriasis, atopic dermatitis, scleroderma, eczema, rosacea, seborrheic dermatitis, melanoma, solar keratosis, ichthyosis, Grover's disease, common warts, keratoacanthoma and/or seborrhoeic keratosis).

In embodiments, the nucleic acid-lipid particle, pharmaceutical composition, or injectate is administered parenterally. Optionally, the nucleic acid-lipid particle, pharmaceutical composition, or injectate is administered via inhalation, topical application or injection. Optionally, the nucleic acid-lipid particle, pharmaceutical composition, or injectate is administered by intravenous injection, intratracheal injection, intra-articular injection, subcutaneous injection, intradermal injection and/or intramuscular injection.

In certain embodiments, the nucleic acid cargo is or includes a synthetic or naturally occurring RNA or DNA, or derivatives thereof. Optionally, the nucleic acid cargo is a modified RNA. Optionally, the modified RNA is a modified mRNA, a modified antisense oligonucleotide or a modified siRNA. Optionally, the modified mRNA encodes a nucleic acid modulating controller.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

The term “lipid” refers to a group of organic compounds that include, but are not limited to, esters of fatty acids and are characterized by being insoluble in water, but soluble in many organic solvents. They are usually divided into at least three classes: (1) “simple lipids” which include fats and oils as well as waxes; (2) “compound lipids” which include phospholipids and glycolipids; (3) “derived lipids” such as steroids.

As used herein, the term “cationic lipid” refers to any of a number of lipid species that carry a net positive charge at a selected pH, such as physiological pH. Cationic lipids include those lipids and salts thereof having one, two, three, or more fatty acid or fatty alkyl chains and a pH-titratable amino head group (e.g., an alkylamino or dialkylamino head group). The cationic lipid is typically protonated (i.e., positively charged) at a pH below the pKa of the cationic lipid and is substantially neutral at a pH above the pKa. The cationic lipids of the description herein may also be termed titratable cationic lipids. In some embodiments, the cationic lipids comprise: a protonatable tertiary amine (e.g., pH-titratable) head group; C18 alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, DOTAP, 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), 1,2-di-γ-linolenyloxy-N,N-dimethylaminopropane (γ-DLenDMA, 1,2-dilinoleyloxy-keto-N,N-dimethyl-3-aminopropane (DLinK-DMA), 1,2-dilinoleyl-4-(2-dimethylaminoethyl)-[1,3]-dioxolane (DLinKC2-DMA) (also known as DLin-C2K-DMA, XTC2, and C2K), 2,2-dilinoleyl-4-(3-dimethylaminopropyl)[1,3]-dioxolane (DLin-K-C3-DMA), 2,2-dilinoleyl-4-(4-dimethylaminobutyl)[1,3]-dioxolane (DLin-K-C4-DMA), 1,2-dilinolenyloxy-4-(2-dimethylaminoethyl)-[1,3]-dioxolane (γ-DLen-C2K-DMA), 1,2-di-γ-linolenyloxy-4-(2-dimethylaminoethyl)-[1,3]-dioxolane (γ-DLen-C2K-DMA), dilinoleylmethyl-3-dimethylaminopropionate (DLin-M-C2-DMA) (also known as MC2), (6Z,9Z,28Z,31Z)-heptatriaconta-6,9,28,31-tetraen-19-yl 4-(dimethylamino) butanoate (DLin-M-C3-DMA) (also known as MC3) and 3-(dilinoleylmethoxy)-N,N-dimethylpropan-1-amine (DLin-MP-DMA) (also known as 1-11). As used herein, “DOTAP,” refers to 1,2-dioleoyl-3-trimethylammonium-propane, or 18:1 TAP, a di-chain, or gemini, cationic lipid.

DOTAP is a cationically charged lipid independent of pH, due to its quaternary structure. It is sold commercially for the liposomal-transfection of DNA, RNA and other negatively charged molecules. In some aspects of the instant disclosure, DOTAP lipid, or variations thereof, combined with C12-200 lipid, or variations thereof, are used in lipid nanoparticles to deliver nucleic acids specifically to the liver. The structure of DOTAP (C₄₂H₈₀NO₄⁺) is shown below:

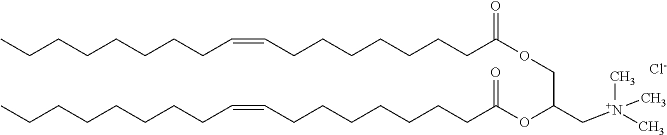 [SMILES: CCCCCCCC/C=C\CCCCCCCC(=O)OCC(C[N+](C)(C)C)OC(=O)CCCCCCC/C=C\CCCCCCCC ; [Cl-]]

As used herein, the term “ionizable lipid” refers to a lipid that becomes cationic (protonated) as the pH is lowered below the pK of the ionizable group of the lipid, but is progressively more neutral at higher pH values. When a component of a lipid-nucleic acid particle, at pH values below the pK, the lipid is then able to associate with negatively charged polynucleic acids. Exemplary ionizable lipids include, without limitation, C12-200; N4-Cholesteryl-Spermine HCl Salt (GL67); N1-[2-((1S)-1-[(3-aminopropyl)amino]-4-[di(3-amino-propyl)amino]butylcarboxamido)ethyl]-3,4-di[oleyloxy]-benzamide (MVL5); 1,2-distearyloxy-N,N-dimethyl-3-aminopropane (DSDMA); 1,2-dioleyloxy-N,N-dimethyl-3-aminopropane (DODMA); 1,2-dilinoleyloxy-N,N-dimethyl-3-aminopropane (DLinDMA); 1,2-dilinolenyloxy-N,N-dimethyl-3-aminopropane (DLenDMA); 1,2-di-7-linolenyloxy-N,N-dimethylaminopropane (7-DLenDMA); 1,2-dilinoleyloxy-keto-N,N-dimethyl-3-aminopropane (DLinK-DMA); 1,2-dilinoleyl-4-(2-dimethylaminoethyl)-[1,3]-dioxolane (DLinKC2-DMA) (also known as DLin-C2K-DMA, XTC2, and C2K); 2,2-dilinoleyl-4-(3-dimethylaminopropyl)[1,3]-dioxolane (DLin-K-C3-DMA); 2,2-dilinoleyl-4-(4-dimethylaminobutyl)[1,3]-dioxolane (DLin-K-C4-DMA); 1,2-dilinolenyloxy-4-(2-dimethylaminoethyl)-[1,3]-dioxolane (γ-DLen-C2K-DMA); 1,2-di-7-linolenyloxy-4-(2-dimethylaminoethyl)-[1,3]-dioxolane (γ-DLen-C2K-DMA); dilinoleylmethyl-3-dimethylaminopropionate (DLin-M-C2-DMA) (also known as MC2); (6Z,9Z,28Z,31Z)-heptatriaconta-6,9,28,31-tetraen-19-yl 4-(dimethylamino) butanoate (DLin-M-C3-DMA) (also known as MC3); 3-(dilinoleylmethoxy)-N,N-dimethylpropan-1-amine (DLin-MP-DMA) (also known as 1-B11); 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA); (2R) 2-({8-[(3β)-cholest-5-en-3-yloxy]octyl}oxy)-N,N-dimethyl-3-[(9Z,12Z)-octadeca-9,12-dien-1-yloxy]propan-1-amine (R-Octyl-CLinDMA); (2S) 2-({8-[(3β)-cholest-5-en-3-yloxy]octyl}oxy)-N,N-dimethyl-3-[(9Z,12Z)-octadeca-9,12-dien-1-yloxy]propan-1-amine (S-Octyl-CLinDMA); (2S)-1-{7-[(3β)-cholest-5-en-3-yloxy]heptyloxy}-3-[(4Z)-dec-4-en-1-yloxy]-N, N-dimethylpropan-2-amine; (2R)-1-{4-[(3β)-cholest-5-en-3-yloxy]butoxy}-3-[(4Z)-dec-4-en-1-yloxy]-N,N-dimethylpropan-2-amine; 1-[(2R)-1-{4-[(3)-cholest-5-en-3-yloxy]butoxy}-3-(octyloxy)propan-2-yl]guanidine; 1-[(2R)-1-{7-[(3β)-cholest-5-en-3-yloxy]heptyloxy}-N,N-dimethyl-3-[(9Z,12Z)-octadeca-9,12-dien-1-yloxy]propan-2-amine; 1-[(2R)-1-{4-[(3β)-cholest-5-en-3-yloxy]butoxy}-N,N-dimethyl-3-[(9Z,12Z)-octadeca-9,12-dien-1-yloxy]propan-2-amine; (2S)-1-({6-[(33))-cholest-5-en-3-yloxy]hexyl}oxy)-N,N-dimethyl-3-[(9Z)-octadec-9-en-1-yloxy]propan-2-amine; (3p)-3-[6-{[(2S)-3-[(9Z)-octadec-9-en-1-yloxyl]-2-(pyrrolidin-1-yl)propyl]oxy}hexyl)oxy]cholest-5-ene; (2R)-1-{4-[(3β)-cholest-5-en-3-yloxy]butoxy}-3-(octyloxy)propan-2-amine; (2R)-1-({8-[(3β)-cholest-5-en-3-yloxy]octyl}oxy)-N,N-dimethyl-3-(pentyloxy)propan-2-amine; (2R)-1-({8-[(3β)-cholest-5-en-3-yloxy]octyl}oxy)-3-(heptyloxy)-N,N-dimethylpropan-2-amine; (2R)-1-({8-[(3β)-cholest-5-en-3-yloxy]octyl}oxy)-N,N-dimethyl-3-[(2Z)-pent-2-en-1-yloxy]propan-2-amine; (2S)-1-butoxy-3-({8-[(3β)-cholest-5-en-3-yloxy]octyl}oxy)-N,N-dimethylpropan-2-amine; (2S-1-({8-[(3β)-cholest-5-en-3-yloxy]octyl}oxy)-3-[2,2,3,3,4,4,5,5,6,6,7,7,8,8,9,9-hexadecafluorononyl)oxy]-N,N-dimethylpropan-2-amine; 2-amino-2-{[(9Z,12Z)-octadeca-9,12-dien-1-yloxy]methyl}propane-1,3-diol; 2-amino-3-({9-[(3β,8ξ,9ξ,14ξ,17ξ,20ξ)-cholest-5-en-3-yloxy]nonyl}oxy)-2-{[(9Z,12Z)-octadeca-9,12-dien-1-yloxy]methyl}propan-1-ol; 2-ammo-3-({6-[(3β, 8ξ,9ξ,14ξ,17ξ,20ξ)-cholest-5-en-3-yloxy]hexyl}oxy)-2-{[(9Z)-octadec-9-en-1-yloxy]methyl}propan-1-ol; (20Z,23Z)-N,N-dimethylnonacosa-20,23-dien-10-amine; (17Z,20Z)-N,N-dimethylhexacosa-17,20-dien-9-amine; (16Z,19Z)-N,N-dimethylpentacosa-16,19-dien-8-amine; (13Z,16Z)-N,N-dimethyldocosa-13,16-dien-5-amine; (12Z,15Z)-N,N-dimethylhenicosa-12,15-dien-4-amine; (14Z,17Z)-N,N-dimethyltricosa-14,17-dien-6-amine; (15Z,18Z)-N,N-dimethyltetracosa-15,18-dien-7-amine; (18Z,21Z)-N,N-dimethylheptacosa-18,21-dien-10-amine; (15Z,18Z)-N,N-dimethyltetracosa-15,18-dien-5-amine; (14Z,17Z)-N,N-dimethyltricosa-14,17-dien-4-amine; (19Z,22Z)-N,N-dimethyloctacosa-19,22-dien-9-amine; (18Z,21Z)-N,N-dimethylheptacosa-18,21-dien-8-amine; (17Z,20Z)-N,N-dimethylhexacosa-17,20-dien-7-amine; (16Z,19Z)-N,N-dimethylpentacosa-16,19-dien-6-amine; (22Z,25Z)-N,N-dimethylhentriaconta-22,25-dien-10-amine; (21Z,24Z)-N,N-dimethyltriaconta-21,24-dien-9-amine; (18Z)-N,N-dimethylheptacos-18-en-10-amine; (17Z)-N,N-dimethylhexacos-17-en-9-amine; (19Z,22Z)-N,N-dimethyloctacosa-19,22-dien-7-amine; N,N-dimethylheptacosan-10-amine; (20Z,23Z)-N-ethyl-N-methylnonacosa-20,23-dien-10-amine; 1-[(11Z,14Z)-1-nonylicosa-11,14-dien-1-yl]pyrrolidine; (20Z)-N,N-dimethylheptacos-20-en-10-amine; (15Z)-N,N-dimethylheptacos-15-en-10-amine; (14Z)-N,N-dimethylnonacos-14-en-10-amine; (17Z)-N,N-dimethylnonacos-17-en-10-amine; (24Z)-N,N-dimethyltritriacont-24-en-10-amine; (20Z)-N,N-dimethylnonacos-20-en-10-amine; (22Z)-N,N-dimethylhentriacont-22-en-10-amine; (16Z)-N,N-dimethylpentacos-16-en-8-amine; (12Z,15Z)-N,N-dimethyl-2-nonylhenicosa-12,15-dien-1-amine; (13Z,16Z)-N,N-dimethyl-3-nonyldocosa-13,16-dien-1-amine; N,N-dimethyl-1-[(1S,2R)-2-octylcyclopropyl]heptadecan-8-amine; 1-[(1S,2R)-2-hexylcyclopropyl]-N,N-dimethylnonadecan-10-amine; N,N-dimethyl-1-[(1S,2R)-2-octylcyclopropyl]nonadecan-10-amine; N,N-dimethyl-21-[(1S,2R)-2-octylcyclopropyl]henicosan-10-amine; N,N-dimethyl-1-[(1S,2S)-2-{[(1R,2R)-2-pentylcyclopropyl]methyl}cyclopropyl]nonadecan-10-amine; N,N-dimethyl-1-[(1S,2R)-2-octylcyclopropyl]hexadecan-8-amine; N,N-dimethyl-1-[(1R,2S)-2-undecylcyclopropyl]tetradecan-5-amine; N,N-dimethyl-3-{7-[(1S,2R)-2-octylcyclopropyl]heptyl}dodecan-1-amine; 1-[(1R,2S)-2-heptylcyclopropyl]-N,N-dimethyloctadecan-9-amine; 1-[(1S,2R)-2-decylcyclopropyl]-N,N-dimethylpentadecan-6-amine; N,N-dimethyl-1-[(1S,2R)-2-octylcyclopropyl]pentadecan-8-amine; and (11E,20Z,23Z)-N,N-dimethylnonacosa-11,20,23-trien-10-amine; as well as pharmaceutically acceptable salts thereof, and stereoisomers of any of the foregoing.

The C12-200 cationic lipid (1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol)) is an ionizable lipid with five tertiary amine structures distributed in a branched, polymer-like structure. High positive net charge of C12-200 can enhance encapsulation of larger RNA molecules into the nanoparticle core. It is sold commercially for the liposomal-transfection of DNA, RNA and other negatively charged molecules. In some aspects of the instant disclosure, DOTAP lipid, or variations thereof, combined with C12-200 lipid, or variations thereof, are used in lipid nanoparticles to deliver nucleic acids specifically to the liver. The structure of C12-200 is shown below:

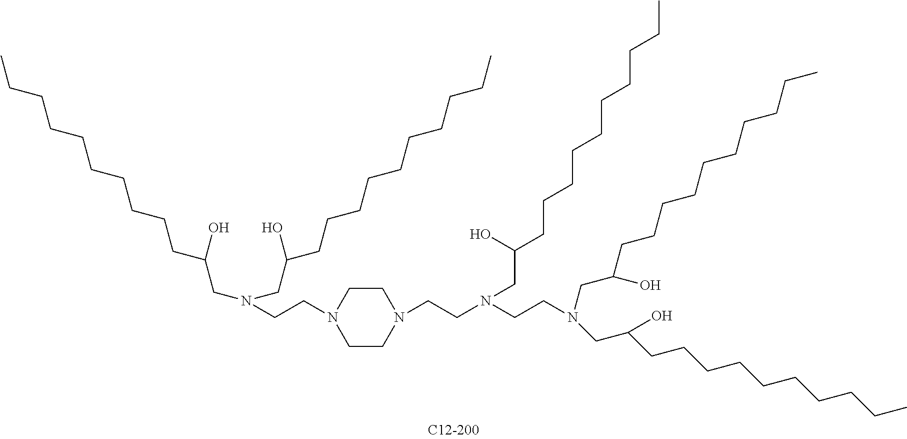 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

As used herein, the term “non-cationic lipid” refers to any neutral lipid, as well as any anionic lipids. A “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides and diacylglycerols. An “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids. In some embodiments, the non-cationic lipid used in the instant disclosure is 1,2-dioleoyl-sn-glycero-3-phosphocholine (DOPC), 1,2-Distearoyl-sn-glycero-3-phosphocholine (DSPC), and/or 1,2-Dioleoyl-sn-glycero-3-phosphoethanolamine (DOPE). In embodiments, the non-cationic lipid is cholesterol (CHE) and/or β-sitosterol.

The term “lipid nanoparticle” as used herein refers to different types of compositions of nano-scale particles, wherein the particles comprising lipids function as carriers across cell membranes and biological barriers and deliver compounds to targeted cells and tissues of humans and other organisms. As used herein, “lipid nanoparticles” of the instant disclosure may further comprise additional lipids and other components. Other lipids may be included for a variety of purposes, such as to prevent lipid oxidation or to attach ligands onto the lipid nanoparticle surface. Any of a number of lipids may be present in lipid nanoparticles of the present disclosure, including amphipathic, neutral, cationic, and anionic lipids. Such lipids can be used alone or in combination, and can also include bilayer stabilizing components such as polyamide oligomers (see, e.g., U.S. Pat. No. 6,320,017), peptides, proteins, detergents, lipid-derivatives, such as PEG coupled to phosphatidylethanolamine and PEG conjugated to ceramides (see, e.g., U.S. Pat. No. 5,885,613).

As used herein, a “PEG” conjugated lipid that inhibits aggregation of particles refers to one or more of a polyethyleneglycol (PEG)-lipid conjugate, a polyamide (ATTA)-lipid conjugate, and a mixture thereof. In one aspect, the PEG-lipid conjugate is one or more of a PEG-dialkyloxypropyl (DAA), a PEG-diacylglycerol (DAG), a PEG-phospholipid, a PEG-ceramide, and a mixture thereof. In one aspect, the PEG-DAG conjugate is one or more of a PEG-dilauroylglycerol (C₁₂), a PEG-dimyristoylglycerol (C₁₄), a PEG-dipalmitoylglycerol (C₁₆), and a PEG-distearoylglycerol (C₁₈). In one aspect, the PEG-DAA conjugate is one or more of a PEG-dilauryloxypropyl (C₁₂), a PEG-dimyristyloxypropyl (C₁₄), a PEG-dipalmityloxypropyl (C₁₆), and a PEG-di stearyloxypropyl (C₁₈). In some embodiments, PEG is 2-dimyristoyl-rac-glycero-3-methoxypolyethylene glycol-2000 (PEG-DMG) and/or 1,2-distearoyl-rac-glycero-3-methoxypolyethylene glycol-2000 (PEG-DSG).

The term “N/P ratio” as used herein refers to the (N)itrogen-to-(P)hosphate ratio between the cationic amino lipid and negatively charged phosphate groups of the nucleic acid.

The “polydispersity index” or “PDI” as used herein is a measure of the heterogeneity of a sample based on size. Polydispersity can occur due to size distribution in a sample or agglomeration or aggregation of the sample during isolation or analysis.

The “zeta potential” or “surface charge” as used herein refers to the degree of electrostatic repulsion between adjacent, similarly charged particles in a dispersion. For molecules and particles that are small enough, a high zeta potential will confer stability, i.e., the solution or dispersion will resist aggregation.

As used herein, the term nucleic acid “cargo” refers to the intended nucleic acid for delivery to the cell or tissue (in embodiments, a therapeutic nucleic acid for delivery to the cell or tissue).

As used herein, the term “nucleic acid-lipid nanoparticle” refers to lipid nanoparticles as described above that associate with or encapsulate one or more nucleic acids to deliver one or more nucleic acid cargoes to a tissue.

As used herein, “encapsulated” can refer to a nucleic acid-lipid nanoparticle formulation that provides a nucleic acid with full encapsulation, partial encapsulation, association by ionic or van der Waals forces, or all of the aforementioned. In one embodiment, the nucleic acid is fully encapsulated in the nucleic acid-lipid nanoparticle.

As used herein, “nucleic acid” refers to a synthetic or naturally occurring RNA or DNA, or derivatives thereof. In one embodiment, a cargo and/or agent of the instant disclosure is a nucleic acid, such as a double-stranded RNA (dsRNA). In one embodiment, the nucleic acid or nucleic acid cargo is a single-stranded DNA or RNA, or double-stranded DNA or RNA, or DNA-RNA hybrid. For example, a double-stranded DNA can be a structural gene, a gene including control and termination regions, or a self-replicating system such as a viral or plasmid DNA. A double-stranded RNA can be, e.g., a dsRNA or another RNA interference reagent. A single-stranded nucleic acid can be, e.g., an mRNA, an antisense oligonucleotide, ribozyme, a microRNA, or triplex-forming oligonucleotide. In certain embodiments, the nucleic acid or nucleic acid cargo may comprise a modified RNA, wherein the modified RNA is one or more of a modified mRNA, a modified antisense oligonucleotide and a modified siRNA. In some embodiments, a nucleic acid cargo of the instant disclosure includes or is a modified mRNA that encodes a nucleic acid modulating controller.

As used herein, the term “modified nucleic acid” refers to any non-natural nucleic acid, including but not limited to those selected from the group comprising 2′-O-methyl modified nucleotides, a nucleotide comprising a 5′-phosphorothioate group, a terminal nucleotide linked to a cholesteryl derivative, a 2′-deoxy-2′-fluoro modified nucleotide, a 5′-methoxy-modified nucleotide (e.g., 5′-methoxyuridine), a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide; internucleoside linkages or backbones including phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

As used herein, the term “nucleic acid modulating controller” refers to a mRNA that encodes for protein controller components, though reference to “nucleic acid modulating controller” can also refer to the mRNA-expressed protein controller components themselves. In certain embodiments, the mRNA-encoded protein controller components include Zinc-Finger proteins (ZFPs) or other forms of DNA or RNA binding domains (DBDs or RBDs) that are associated with (and optionally tethered to) one or more epigenetic regulators or nucleases (the epigenetic regulators or nucleases are generally referred to as effectors, effector domains, or effector moieties). Without wishing to be bound by theory, an advantage of a nucleic acid modulating controller as described herein is that it provides durable gene programming only at the confluence of (1) where the nucleic acid modulating controller-encoding mRNA is expressed, (2) where nucleic acid binding of the ZFP or other nucleic acid binding domain occurs and (3) where the associated effector domain is able to exert activity (i.e. where the effector domain is capable of changing the epigenomic state (e.g., in the instance of an epigenomic controller)).

As used herein, the term “effector moiety” or “effector domain” refers to a domain that is capable of altering the expression of a target gene when localized to an appropriate site in a cell, e.g., in the nucleus of a cell. In some embodiments, an effector moiety recruits components of the transcription machinery. In some embodiments, an effector moiety inhibits recruitment of components of transcription factors or expression repressing factors. In some embodiments, an effector moiety comprises an epigenetic modifying moiety (e.g., epigenetically modifies a target DNA sequence). Specific examples of effector moieties include, without limitation, effectors capable of binding Krueppel-associated box (KRAB) domains (KRAB is a domain of around 75 amino acids that is found in the N-terminal part of about one third of eukaryotic Krueppel-type C2H2 zinc finger proteins (ZFPs)) and the engineered prokaryotic DNA methyltransferase MQ1, among others.

As used herein, “epigenetic modifying moiety” refers to a domain that alters: i) the structure, e.g., two dimensional structure, of chromatin; and/or ii) an epigenetic marker (e.g., one or more of DNA methylation, histone methylation, histone acetylation, histone sumoylation, histone phosphorylation, and RNA-associated silencing), when the epigenetic modifying moiety is appropriately localized to a nucleic acid (e.g., by a targeting moiety). In some embodiments, an epigenetic modifying moiety comprises an enzyme, or a functional fragment or variant thereof, that affects (e.g., increases or decreases the level of) one or more epigenetic markers. In some embodiments, an epigenetic modifying moiety comprises a DNA methyltransferase, a histone methyltransferase, CREB-binding protein (CBP), or a functional fragment of any thereof.

As used herein, the term “expression control sequence” refers to a nucleic acid sequence that increases or decreases transcription of a gene, and includes (but is not limited to) a promoter and an enhancer. An “enhancing sequence” refers to a subtype of expression control sequence and increases the likelihood of gene transcription. A “silencing or repressor sequence” refers to a subtype of expression control sequence and decreases the likelihood of gene transcription.

As used herein, the term “expression repressor” refers to an agent or entity with one or more functionalities that decreases expression of a target gene in a cell and that specifically binds to a DNA sequence (e.g., a DNA sequence associated with a target gene or a transcription control element operably linked to a target gene). In certain embodiments, an expression repressor comprises at least one targeting moiety and optionally one effector moiety.

As used herein, the term “targeting moiety” means an agent or entity that specifically targets, e.g., binds, a genomic sequence element (e.g., an expression control sequence or anchor sequence; promoter, enhancer or CTCF site). In some embodiments, the genomic sequence element is proximal to and/or operably linked to a target gene (e.g., MYC).

As used herein, “liver tissue” may refer to any cell or population of cells within the organ of the liver including but not limited to hepatocytes, vascular cells, endothelial cells, parenchymal cells, non-parenchymal cells, fibroblasts, mesenchymal cells, immune cells, cancer cells, Kupffer cells, astrocytes, oval-shaped vascular endothelial cells, and liver-derived stem/progenitor cells. In certain embodiments, the nucleic acid-lipid nanoparticle targets liver tissue. In some other embodiments, the nucleic acid-lipid nanoparticle may target other cells or tissues including but not limited to brain, nerve, skin, eye, pharynx, larynx, heart, vascular, hematopoietic (e.g., white blood cell or red blood cell), breast, lung, pancreas, spleen, esophagus, gall bladder, stomach, intestine, colon, kidney, urinary bladder, ovary, uterus, cervix, prostate, muscle, bone, thyroid, parathyroid, adrenal, and pituitary cells or tissues.

As used herein, “localization” refers to the position of a lipid, peptide, or other component of a lipid particle of the instant disclosure, within an organism and/or tissue. In some embodiments, localization can be detectible in individual cells. In some embodiments a label can be used for detecting localization, e.g., a fluorescent label, optionally a fluorescently labeled lipid, optionally Cy7. In some embodiments, the label of the lipid nanoparticle may be a quantum dot, or the lipid detectible by stimulated Raman scattering. In other embodiments, the label is any fluorophore known in the art, i.e. with excitation and emission in the ultraviolet, visible, or infrared spectra. In some embodiments the localization is detected or further corroborated by immunohistochemistry or immunofluorescence.

As used herein, the term “activity” refers to any detectable effect that is mediated by a component or composition of the instant disclosure. In embodiments, “activity” as used herein, can refer to a measurable (whether directly or by proxy) effect, e.g., of a cargo of the instant lipid particles of the disclosure. Examples of activity include, without limitation, the intracellular expression and resulting effect(s) of a nucleic acid cargo (e.g., a mRNA, a CRISPR/Cas system, a RNAi agent, a nucleic acid modulating controller, etc.), which can optionally be measured at a cellular, tissue, organ and/or organismal level.

As used herein, “accelerated blood clearance” or “ABC” refers to a well-documented phenomenon caused by immune system activation against PEG molecules on the surface of LNPs. ABC is responsible for the clearance of nanoparticles from systemic circulation upon repeated dosing. In some embodiments, lipid particles of the instant disclosure can avoid or reduce accelerated blood clearance of lipid particles, by employing PEG-free formulations, which can also provide for improved (e.g., less toxic and/or more effective) repeated systemic administration of such lipid particles. As used herein, “multidosing” refers to two or more doses of a lipid nanoparticle formulation given as part of a therapeutic regimen to a subject.

As used herein, the term “liver disease or disorder” may include, without limitation, a disease or disorder such as hepatitis A, hepatitis B, hepatitis C, fatty liver disease, liver cirrhosis, liver cancer (e.g., hepatocellular carcinoma), hemochromatosis, and Wilson disease.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, “administration” to a subject may include parenteral administration, optionally for intravenous injection, inhalation, intravenous, intra-arterial, intratracheal, topical, or involve direct injection into a tissue.

The term “treating” includes the administration of compositions to prevent or delay the onset of the symptoms, complications, or biochemical indicia of a disease (e.g., cancer, including, e.g., tumor formation, growth and/or metastasis), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

As used herein, a “pharmaceutical composition” comprises a pharmacologically effective amount of a lipid particle, optionally a nucleic-acid lipid nanoparticle (NLNP) and a pharmaceutically acceptable carrier. As used herein, “pharmacologically effective amount,” “therapeutically effective amount” or simply “effective amount” refers to that amount of nucleic acid effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to induce at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A-1C demonstrate formulation and properties of an initial mixed lipid particle prepared with a validated ionizable lipid and a distinct multivalent cationic polymer. FIG. 1A shows a table of the components making up this initial mixed particle, including DOPC, MC3, bPEI, CHE, and PEG2k-DMG, as well as characterization results of the mixed lipid particle, including particle size, zeta potential, and polydispersity index. FIG. 1B shows post-transfection images of Hepa 1-6 cells after being treated continuously with the initial mixed lipid particle for a period of 48 hours. FIG. 1C presents a graph demonstrating dose-response of cell viability (%) observed for Hepa 1-6 cells across different added mixed lipid particle volumes (10 μl, 5 μl, and 2.5 μl).

FIGS. 2A-2C show that mixed lipid particles transfected nucleic acid cargoes into traditionally hard-to-transfect cell populations. FIG. 2A presents a table that lists the components of “MbP-2” mixed lipid particles, which included DOPC, MC3, bPEI, CHE, and PEG2k-DMG at indicated levels, as well as of a MC3-only lipid particle which included DOPC, MC3, CHE, and PEG2k-DMG. FIG. 2B shows plots of GFP and Cy5 signal expression levels observed across populations of Hepa 1-6 cells respectively treated with a control formulation, a formulation of mixed lipid (MbP-2) nanoparticles, and a formulation of MC3-only lipid nanoparticles. Notably, effective GFP mRNA transfection was observed in more than 50% of cells treated with mixed lipid (MbP-2) nanoparticles, as compared to a 16% GFP mRNA transfection rate for cells treated with MC3-only lipid nanoparticles. FIG. 2C shows the GFP signal expression distributions of such transfected cell populations. Notably, the mixed lipid nanoparticles produced, on average, 1.5-fold increased GFP signal in transfected cells as compared to MC3-only lipid nanoparticles, while such observed GFP levels in mixed lipid nanoparticle-treated cells were also, on average, 20-fold higher than GFP signal levels in control-treated cells.

FIGS. 3A-3D show the effects of DOTAP/C12-200 (DC) mixed lipid nanoparticles (LNPs) upon cell viability in LNP-treated A549 human lung cancer cells. FIG. 3A shows a table of the components and evaluated characteristics of different formulations (Mix C to Mix N) of mixed lipid particles. Mixed lipid particles C to N included C12-200, DOTAP, DOPE, Cholesterol (CHE), and (with Mix J being the lone exception) PEG2k-DMG. FIG. 3B shows a histogram that depicts observed cell viability of A549 human lung cancer cells following treatment with different mixed lipid nucleic acid-lipid particles, including Mix C, Mix D, Mix E, Mix F, Mix G and a control formulation, where the percentage molarity of C12-200 in the mixed lipid particle formulations was maintained at 18% while the percentages of DOTAP and DOPE in these mixed lipid formulations were altered. FIG. 3C shows a histogram that depicts observed cell viability of A549 human lung cancer cells following treatment with different mixed lipid nucleic acid-lipid particles, including Mix C, Mix H, Mix I, and a control formulation, where the percentage molarity of C12-200 in the mixed lipid particle formulations was maintained at 18% while the percentage of PEG-lipid in these mixed lipid formulations was altered. FIG. 3D shows a histogram that depicts observed cell viability of A549 human lung cancer cells following treatment with different mixed lipid nucleic acid-lipid particles, including Mix K, Mix L, Mix M, Mix N, and a control formulation, where the percentage molarity of C12-200 in the mixed lipid particle formulations was maintained at 18% while the percentages of cholesterol relative to DOTAP in these mixed lipid formulations were altered.

FIGS. 4A-4C show the transfection efficacy of DOTAP/C12-200 (DC) mixed lipid nanoparticles (LNPs) in LNP-treated A549 human lung cancer cells. FIG. 4A shows a histogram that depicts observed transfected reporter mCherry signal in A549 human lung cancer cells treated with different mixed lipid nucleic acid-lipid particles, including Mix C, Mix D, Mix E, Mix F, Mix G and a control formulation, as listed in FIG. 3A above. FIG. 4B shows a histogram that depicts observed transfected reporter mCherry signal in A549 human lung cancer cells treated with different mixed lipid nucleic acid-lipid particles, including Mix C, Mix H, Mix I and a control formulation, as listed in FIG. 3A above. FIG. 4C shows a histogram that depicts observed transfected reporter mCherry signal in A549 human lung cancer cells treated with different mixed lipid nucleic acid-lipid particles, including Mix K, Mix L, Mix M, Mix N, and a control formulation, as listed in FIG. 3A above.

FIG. 5 shows a microscopic image of A549 human lung cancer cells treated with Mix C at 0.16 μg/ml, where blue signal indicates nuclei, while red signal indicates transfected mCherry reporter expression. Notably, all cells of the image clearly expressed the mCherry reporter, even at a low transfected mRNA concentration of 0.16 μg/ml.

FIGS. 6A and 6B show that mixed lipid particles were stable under different storage conditions. FIG. 6A shows a table of mixed lipid particle properties before and after 35-day storage at 4° C. or −80° C., stored in solutions containing water only, water with 10% sucrose, HEPES buffer only and HEPES buffer with 10% sucrose, respectively. FIG. 6B shows an image of gel electrophoresis performed upon mixed lipid particles harboring a RNA cargo, stored in water at 4° C. (columns 1 and 2) or −80° C. (columns 4 and 5). Columns 2 and 5 were treated with 2% Triton X-100, which disrupted the particles and allowed free RNA to run across the gel. Notably, all formulations remained stable at 4° C., whereas only mixed lipid particles with cryoprotectant (10% sucrose) had their characteristics preserved at −80° C.

FIGS. 7A-7D show that administration of mixed lipid particles harboring mCre mRNA to mice harboring a nuclear Cre-activated tdTomato (tdTom) reporter resulted in selective delivery and tdTom production in the liver. FIG. 7A shows a table of the components and properties of two different mixed lipid particles employed to deliver the mCre mRNA in vivo, where component “C” stands for C12-200. FIG. 7B shows a plot of expressed tdTom signal observed in different organs (liver, lung and spleen) for both mixed lipid mCre mRNA particles (administered at 1 mg/kg), as compared to a MC3-only mCre mRNA particle as control (administered at 3 mg/kg). FIG. 7C shows representative images of tdTom signal in different organs. Notably, highly specific liver activity was observed for both mixed lipid particles tested. FIG. 7D shows a plot of tdTom signal intensity in liver, lungs and spleen, normalized for area, which revealed that the mixed lipid mCre mRNA particles transfected liver cells in vivo at 80-100% higher rates than the MC3-only mCre mRNA lipid particle control.

FIG. 8 shows the cellular association in the liver of tested mixed lipid particles (as described in FIG. 7A above). Immunohistochemistry images of liver samples are displayed, which confirmed tdTomato production and Cy7-labeled particle accumulations for both tested particles (both stained in brown).

FIGS. 9A-9F show plots presenting liver function safety evaluation tests of mixed lipid and DOTAP particles in mice. Liver function tests performed included: measurement of alkaline phosphatase (ALP) levels (FIG. 9A), alanine transaminase (ALT) levels (FIG. 9B), aspartate transaminase (AST) levels (FIG. 9C), direct bilirubin (DBILI) levels (FIG. 9D), total bilirubin (TBILI) levels (FIG. 9E), and lactate dehydrogenase (LDH) levels (FIG. 9F). Notably, mixed lipid particles of the instant disclosure were identified as safe at 1 mg/kg mRNA dose.

FIGS. 10A and 10B show that mixed lipid particles harboring a nucleic acid modulating controller (known to induce VEGFa expression) robustly increased serum VEGFa levels when administered to mice. FIG. 10A presents a histogram of observed serum VEGFa levels following administration of mixed lipid particles DC20182 and DC50182 harboring the VEGFa-modulating controller, across indicated dosed concentrations, as compared to PBS-treated control mice. FIG. 10B presents a histogram that displays the observed percentage change in VEGFa levels over PBS control levels, across the nucleic acid-lipid particles of FIG. 10A above. Notably, intravenous administration of a VEGFa-modulating nucleic acid controller in mixed lipid particles to mice at three distinct dose levels provoked significantly increased serum VEGFa levels, especially at a 1 mg/kg dose level, as compared to control-treated mice.

### DETAILED DESCRIPTION OF THE INVENTION

The instant disclosure provides, at least in part, mixed cationic lipid particle compositions, formulations and associated methods, for delivery of lipid particle-associated molecular cargoes to the cells of a subject. In certain aspects, nucleic-acid lipid nanoparticles are provided that preferentially localize to and deliver associated nucleic acid cargoes to the liver of a subject, with delivery occurring to various types of tissue within the liver of a subject. In particular, the particles of the instant disclosure include mixtures of ionizable lipid and one or more distinct cationic lipids, while also in certain embodiments including a non-cationic “helper” lipid, a structural lipid (e.g., cholesterol, β-sitosterol or derivatives thereof) and/or a stabilizer/anti-aggregation lipid (e.g., PEG-lipid).

Nucleic acid therapy has well-known, tremendous potential to treat diseases at the gene level. However, safe and effective delivery systems are essential for nucleic acid therapeutics. Non-specific delivery to organs and tissues often results in off-site effects and toxicity. Delivery of therapeutics to a specific organ of interest is a well-recognized need in the development of lipid-nanoparticles, as well as in drug development generally. The concept of only targeting the cause of a disease without harming other parts of the body was described by Ehrlich 120 years ago. However, extant methods do not provide defined or well-known methodologies for developing nanoparticles targeting specific tissues without introducing additional ligand-based targeting strategies. Organ-specific targeting of lipid nanoparticles based on the structural affinity of the lipid to the tissue, as now disclosed herein, therefore meets a well-established need in terms of reducing off-site effects and toxicity.

Traditional LNPs are composed of four main components. An ionizable or cationic lipid for mRNA encapsulation, amphipathic helper phospholipids for increased efficacy, cholesterol for structural stability and PEG-lipids for steric stability. This first generation of LNPs can be considered as “one ionizable lipid-only LNPs”, or “single LNPs”. Conventionally, effective intracellular delivery materials have relied on an optimal balance of ionizable amines to bind and release RNAs (pKa between 6.0 and 6.5) and nanoparticle-stabilizing hydrophobicity. Thus, there has been an exhaustive focus on developing ionizable lipids, which have been proven to be highly effective delivery platforms for liver and hepatocytes. However, changing the chemical structure of the ionizable/cationic lipid to achieve different pKa values and generating libraries, although validated, is a time consuming, investment heavy and labor-intensive exercise.

It was initially contemplated herein that using more than one cationic lipid might generate unique LNPs possessing useful properties for nucleic acid delivery, by fine tuning the pKa of the overall system for increased intracellular delivery efficacy, and might also change the tropism of the single LNPs by structural affinity of the additional lipid. In initial studies of the instant disclosure, mixed ionizable lipid/cationic polymer formulations were prepared and were identified to possess size and other characteristics (including low cellular toxicity in vitro) amenable to use of such particles as nucleic acid-lipid nanoparticle delivery modalities. Mixed lipid particles that combined an ionizable lipid with a multivalent cationic polymer (branched polyethyleneimine, bPEI) were then discovered to exhibit similar characteristics and were also identified to be capable of effective transfection of even large nucleic acid modulating controllers (e.g., a mRNA that encodes protein controller components) to traditionally hard-to-transfect cell populations (e.g., primary T-cells).

DOTAP, a well-known quaternary amino lipid, is a structural component of certain nucleic acid-lipid nanoparticles (LNPs) that were recently described to exhibit lung-specific nucleic acid delivery without requiring a further active-targeting component in such LNPs. Lipid particles that included both ionizable lipid and DOTAP as mixed cationic lipids were then prepared and assessed for efficacy of nucleic acid cargo delivery, as well as for whether tissue-specific delivery would be observed (without a priori knowledge of whether and/or which tissue specificity/specificities might be identified). C12-200 was selected as the ionizable lipid for such exemplified DOTAP-containing mixed lipid formulations, though it is expressly contemplated that other ionizable lipids might replace C12-200 while still yielding effective particles. In exemplified embodiments, the non-cationic “helper lipid” employed was DOPE (1,2-Dioleoyl-sn-glycero-3-phosphoethanolamine), which was selected for its described ability to improve endosomal escape by fusing with the endosomal membrane due to its ability to form an inverse hexagonal phase. It has been previously identified that adding saturation into unsaturated lipid structures in LNPs improves the intracellular activity of such LNPs. Meanwhile, cholesterol and PEG2k-DMG levels have been maintained in currently exemplified particles at levels similar to those in previously described particles.

The data presented herein have specifically identified that C12-200 addition into DOTAP-based LNPs changes the tropism of such formulations from lungs to liver, while thereby also demonstrating organ-selective activity of such mixed LNPs upon systemic administration. Briefly, it was previously reported that LNPs with 50% mol DOTAP—independent from their particle size, surface charge and PEGylation—provided robust lung targeting that was attributed to structural affinity of DOTAP for lung tissues. The instant disclosure has discovered specific activity of C12-200/DOTAP mixed lipid particles in the liver, confirmed herein by reporter-based biodistribution and also efficacy studies. Moreover, a library of mixed lipid particle formulations that employ a low level of C12-200 (to mitigate possible toxicity of C12-200) have been described herein. When present at 18% in the mixed lipid particles, C12-200 was able to provide a liver-targeting effect, without the need for active targeting ligands.

As noted above, C12-200 carries a high positive net charge, which can enhance encapsulation of larger RNA molecules into a nanoparticle's core (as compared to other cationic lipids such as DOTAP and MC3, which present only one amine per molecule). Strong RNA/C12-200 packing can therefore allow for fine tuning of the remaining lipid components and surface charge of a C12-200-containing particle, without significantly affecting the overall diameter of such a particle. Without wishing to be bound by theory, high positive net charge of C12-200-containing particles at endosomal acidic pH can also improve cargo delivery into the cytoplasm of targeted cells, attributable to a proton sponge effect. Such particles can therefore provide efficient mRNA transfection in lower treatment dosing. The particles of the instant disclosure also provide a basis for developing larger libraries of mixed lipid particles, which, by fine-tuning the components of such mixed lipid particles, might now be expected to allow for different organ/tissue targeting than even those currently described (most notably, respectively lung-specific and liver-specific particles that can be administered parenterally), without the need for employment of an active targeting ligand.

Thus, the instant disclosure provides, without limitation, the following features and benefits: (1) Altering the primary target organ for DOTAP-based particles from lung to liver via introduction of an ionizable lipid (C12-200 as specifically exemplified herein); (2) Liver-specific delivery of nucleic acid cargoes/therapeutics via use of such DOTAP-based particles; (3) Prevention of off-target toxicity (including reduction/prevention of toxicity by maintaining exemplified C12-200 levels below about 20%, which is the level above which C12-200-related cytotoxic effects might be expected to occur); (4) Increased efficacy upon systemic administration (by IV, as exemplified), thereby providing an improved alternative to widely-used liver targeting LNPs that employ DLin-MC3-DMA; and (5) The process for particle formation is both scalable and consistent, allowing for possible use in human therapeutic formulations for direct administration.

Although the DOTAP/ionizable lipid particles of the instant disclosure have been identified to selectively deliver cargoes to liver tissue, in some aspects, delivery to other regions having leaky or fenestrated capillaries, such as joint and/or inflammation sites, and/or the spleen, with the DOTAP/ionizable lipid particles of the instant disclosure or variations thereof, is also contemplated. In addition, administration of the particles of the instant disclosure via inhalation, topical application or non-intravenous injection is also expressly contemplated. Without limitation, a nucleic acid-lipid particle, pharmaceutical composition, or injectate of the instant disclosure can be administered via a non-intravenous route, e.g., by intratracheal injection, intra-articular injection, subcutaneous injection, intradermal injection and/or intramuscular injection.

Various expressly contemplated components of certain compositions and methods of the instant disclosure are considered in additional detail below.

### DOTAP C12-200-Based Lipid Nanoparticle (“DC LNP”) Compositions

1,2-dioleoyl-3-trimethylammonium-propane, DOTAP, or 18:1 TAP is a cationic lipid. DOTAP is cationically charged independent of pH, due to its quaternary structure. The structure of DOTAP (C₄₂H₈₀NO₄⁺) is presented above.

C12-200 (1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol)) is an ionizable lipid with five tertiary amine structures distributed in a branched, polymer-like structure. The structure of C12-200 has also been presented above.

In certain embodiments of the lipid particles of the instant disclosure, and in related methods of the instant disclosure, at least about 5%, at least about 10%, at least about 20%, at least about 30%, at least about 40%, at least about 50%, or between about 10% and 50% (molar basis) of the total lipids present in a lipid nanoparticle of the disclosure are DOTAP. In certain embodiments of the lipid particles of the instant disclosure, and in related methods of the instant disclosure, at least about 5%, at least about 10%, at least about 15%, at least about 20%, between about 5% and about 20%, less than about 20%, between about 12% and about 45%, or about 18% (molar basis) of the total lipids are an ionizable lipid (e.g., C12-200). In certain embodiments of the lipid particles of the instant disclosure, and in related methods of the instant disclosure, at least about 0.1%, at least about 5%, at least about 10%, at least about 20%, at least about 40%, at least about 60%, between about 24% and about 64%, about 24%, about 34%, about 44%, about 54%, or about 64% (molar basis) of the total lipids are cholesterol, 0-sitosterol, and/or derivatives thereof. In certain embodiments, at least about 0.1%, at least about 5%, at least about 10%, at least about 20%, at least about 40%, between about 6% and about 46%, about 6%, about 7%, about 7.5%, about 8%, about 16%, about 26%, about 36% or about 46% (molar basis) of the total lipids are other non-cationic lipids, e.g. DOPE, DOPC and/or DSPC. In certain embodiments of the lipid particles of the instant disclosure, and in related methods of the instant disclosure, the particle includes a conjugated lipid that inhibits aggregation of particles present at 0.01 mol % to about 3 mol %, at about 0.5 mol %, at about 1.0 mol %, at about 1.5 mol %, or at about 2.0 mol % of the total lipid present. Examples of such conjugated lipids include, without limitation, polyethyleneglycol (PEG)-lipid conjugates, e.g., PEG2000-lipid conjugates, e.g., 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k).

Lipid nanoparticles of any size may be used according to the instant disclosure. In certain embodiments of the instant disclosure, lipid nanoparticles have a size ranging from about 0.02 microns to about 0.4 microns, between about 0.05 and about 0.2 microns, or between 0.07 and 0.12 microns in diameter.

In some embodiments, the LNPs may also comprise other cationic lipids including but not limited to, those comprising a protonatable tertiary amine (e.g., pH-titratable) head group; C18 alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), 1,2-di-γ-linolenyloxy-N,N-dimethylaminopropane (γ-DLenDMA, 1,2-dilinoleyloxy-keto-N,N-dimethyl-3-aminopropane (DLinK-DMA), 1,2-dilinoleyl-4-(2-dimethylaminoethyl)-[1,3]-dioxolane (DLinKC2-DMA) (also known as DLin-C2K-DMA, XTC2, and C2K), 2,2-dilinoleyl-4-(3-dimethylaminopropyl)[1,3]-dioxolane (DLin-K-C3-DMA), 2,2-dilinoleyl-4-(4-dimethylaminobutyl)[1,3]-dioxolane (DLin-K-C4-DMA), 1,2-dilinolenyloxy-4-(2-dimethylaminoethyl)-[1,3]-dioxolane (γ-DLen-C2K-DMA), 1,2-di-γ-linolenyloxy-4-(2-dimethylaminoethyl)-[1,3]-dioxolane (γ-DLen-C2K-DMA), dilinoleylmethyl-3-dimethylaminopropionate (DLin-M-C2-DMA) (also known as MC2), (6Z,9Z,28Z,31Z)-heptatriaconta-6,9,28,31-tetraen-19-yl 4-(dimethylamino) butanoate (DLin-M-C3-DMA) (also known as MC3) and 3-(dilinoleylmethoxy)-N,N-dimethylpropan-1-amine (DLin-MP-DMA) (also known as 1-B11).

In some embodiments, the particles of the instant disclosure may include neutral lipids, for example, diacylphosphatidylcholine, diacylphosphatidylethanolamine, ceramide, sphingomyelin, cephalin, cholesterol, cerebrosides and diacylglycerols. In other embodiments, LNPs may include anionic lipids, including but not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids. In some aspects, the non-cationic lipid used in the instant disclosure is 1,2-Dioleoyl-sn-glycero-3-phosphoethanolamine (DOPE), 1,2-dioleoyl-sn-glycero-3-phosphocholine (DOPC), and/or 1,2-Distearoyl-sn-glycero-3-phosphocholine (DSPC). In some aspects, one or more non-cationic lipid of the instant particles is cholesterol (CHE), 0-sitosterol, and/or derivatives thereof.

In some embodiments that employ PEG-conjugated lipids, the PEG-conjugated lipid is one or more of a polyethyleneglycol (PEG)-lipid conjugate, a polyamide (ATTA)-lipid conjugate, and a mixture thereof. In one aspect, the PEG-lipid conjugate is one or more of a PEG-dialkyloxypropyl (DAA), a PEG-diacylglycerol (DAG), a PEG-phospholipid, a PEG-ceramide, and a mixture thereof. In one aspect, the PEG-DAG conjugate is one or more of a PEG-dilauroylglycerol (C₁₂), a PEG-dimyristoylglycerol (C₁₄), a PEG-dipalmitoylglycerol (C₁₆), and a PEG-distearoylglycerol (C₁₈). In one aspect, the PEG-DAA conjugate is one or more of a PEG-dilauryloxypropyl (C₁₂), a PEG-dimyristyloxypropyl (C₁₄), a PEG-dipalmityloxypropyl (C₁₆), and a PEG-di stearyloxypropyl (C₁₈). In some embodiments, PEG is 2-dimyristoyl-rac-glycero-3-methoxypolyethylene glycol-2000 (PEG-DMG) and/or 1,2-distearoyl-rac-glycero-3-methoxypolyethylene glycol-2000 (PEG-DSG).

In some embodiments, amphipathic lipids are included in particles of the instant disclosure. Amphipathic lipids may refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and β-acyloxyacids, can also be used. Additionally, such amphipathic lipids can be readily mixed with other lipids, such as triglycerides and sterols.

Also suitable for inclusion in the lipid particles of the instant disclosure are programmable fusion lipid formulations. Such formulations have little tendency to fuse with cell membranes and deliver their cargo until a given signal event occurs. This allows the lipid formulation to distribute more evenly after injection into an organism or disease site before it starts fusing with cells. The signal event can be, for example, a change in pH, temperature, ionic environment, or time. In the latter case, a fusion delaying or “cloaking” component, such as an ATTA-lipid conjugate or a PEG-lipid conjugate, can simply exchange out of the lipid nanoparticle membrane over time. By the time the formulation is suitably distributed in the body, it has lost sufficient cloaking agent so as to be fusogenic. With other signal events, it is desirable to choose a signal that is associated with the disease site or target cell, such as increased temperature at a site of inflammation.

In certain embodiments, it can be desirable to target the lipid particles of this disclosure further, using targeting moieties that are specific to a cell type or tissue. Targeting of lipid nanoparticles using a variety of targeting moieties, such as ligands, cell surface receptors, glycoproteins, vitamins (e.g., riboflavin) and monoclonal antibodies, has been previously described (see, e.g., U.S. Pat. Nos. 4,957,773 and 4,603,044). The targeting moieties can comprise the entire protein or fragments thereof.

Targeting mechanisms generally require that the targeting agents be positioned on the surface of the lipid nanoparticle in such a manner that the target moiety is available for interaction with the target, for example, a cell surface receptor. A variety of different targeting agents and methods are known and available in the art, including those described, e.g., in Sapra, P. and Allen, T M, Prog. Lipid Res. 42(5):439-62 (2003); and Abra, R M et al., J. Lipid nanoparticle Res. 12:1-3, (2002).

Standard methods for coupling target agents can be used. For example, phosphatidylethanolamine, which can be activated for attachment of target agents, or derivatized lipophilic compounds, such as lipid-derivatized bleomycin, can be used. Antibody-targeted lipid nanoparticles can be constructed using, for instance, lipid nanoparticles that incorporate protein A (see, Renneisen, et al., J. Bio. Chem., 265:16337-16342 (1990) and Leonetti, et al., Proc. Natl. Acad. Sci. (USA), 87:2448-2451 (1990). Other examples of antibody conjugation are disclosed in U.S. Pat. No. 6,027,726, the teachings of which are incorporated herein by reference. Examples of targeting moieties can also include other proteins, specific to cellular components, including antigens associated with neoplasms or tumors. Proteins used as targeting moieties can be attached to the lipid nanoparticles via covalent bonds (see, Heath, Covalent Attachment of Proteins to Lipid nanoparticles, 149 Methods in Enzymology 111-119 (Academic Press, Inc. 1987)). Other targeting methods include the biotin-avidin system.

A variety of methods for preparing lipid nanoparticles are known in the art, including e.g., those described in Szoka, et al., Ann. Rev. Biophys. Bioeng., 9:467 (1980); U.S. Pat. Nos. 4,186,183, 4,217,344, 4,235,871, 4,261,975, 4,485,054, 4,501,728, 4,774,085, 4,837,028, 4,946,787; PCT Publication No. WO 91/17424; Deamer and Bangham, Biochem. Biophys. Acta, 443:629-634 (1976); Fraley, et al., Proc. Natl. Acad. Sci. USA, 76:3348-3352 (1979); Hope, et al., Biochem. Biophys. Acta, 812:55-65 (1985); Mayer, et al., Biochem. Biophys. Acta, 858:161-168 (1986); Williams, et al., Proc. Natl. Acad. Sci., 85:242-246 (1988); Lipid nanoparticles, Marc J. Ostro, ed., Marcel Dekker, Inc., New York, 1983, Chapter 1; Hope, et al., Chem. Phys. Lip., 40:89 (1986); and Lipid nanoparticles: A Practical Approach, Torchilin, V. P. et al., ed., Oxford University Press (2003), and references cited therein. Suitable methods include, but are not limited to, sonication, extrusion, high pressure/homogenization, microfluidization, detergent dialysis, calcium-induced fusion of small lipid nanoparticle vesicles, and ether-infusion methods, all of which are well known in the art.

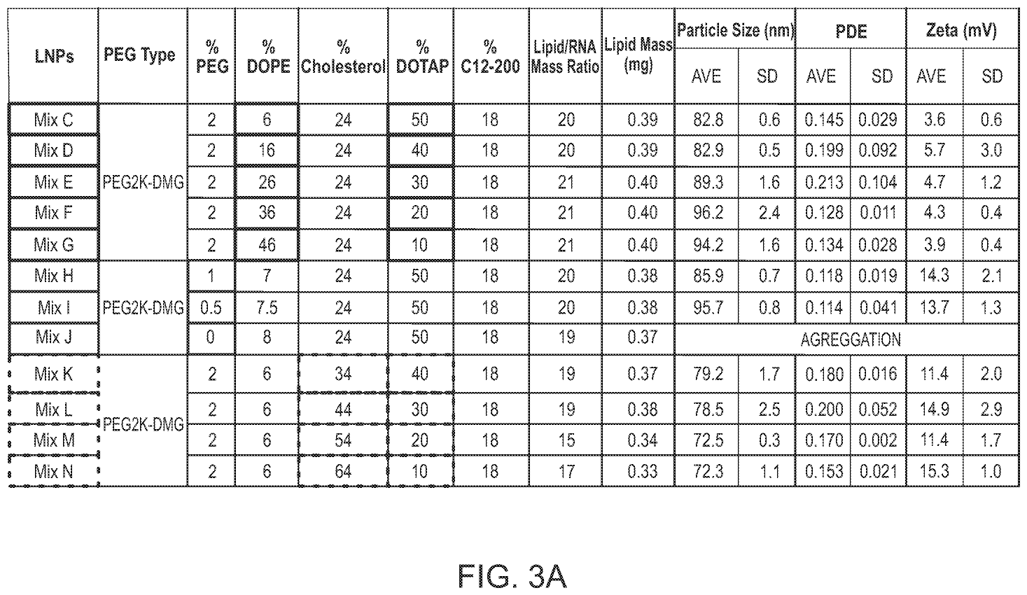

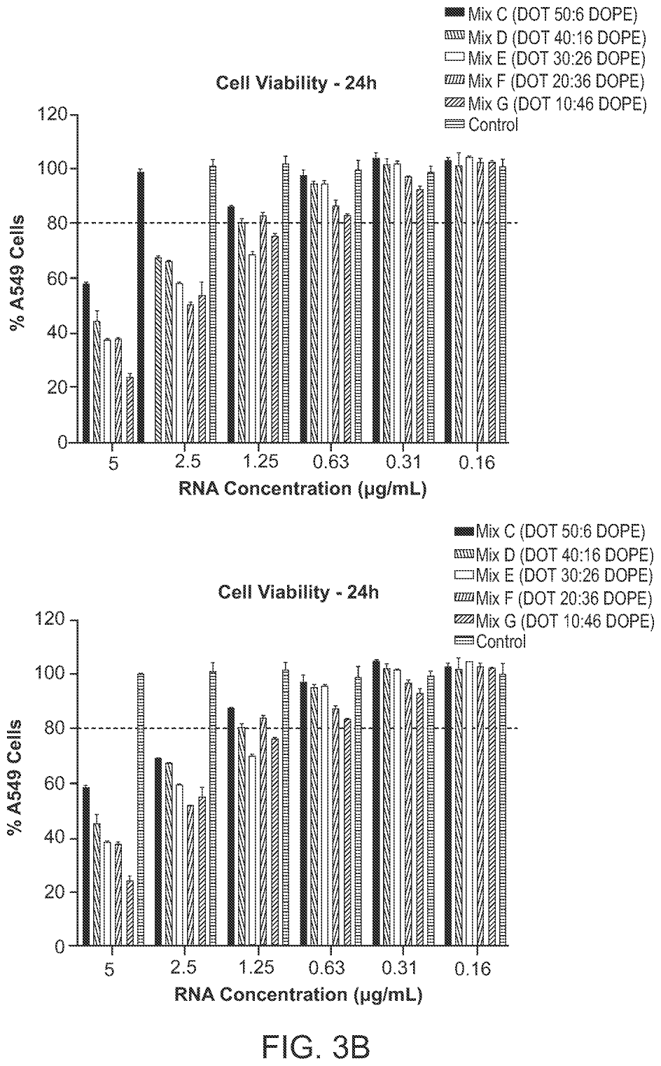

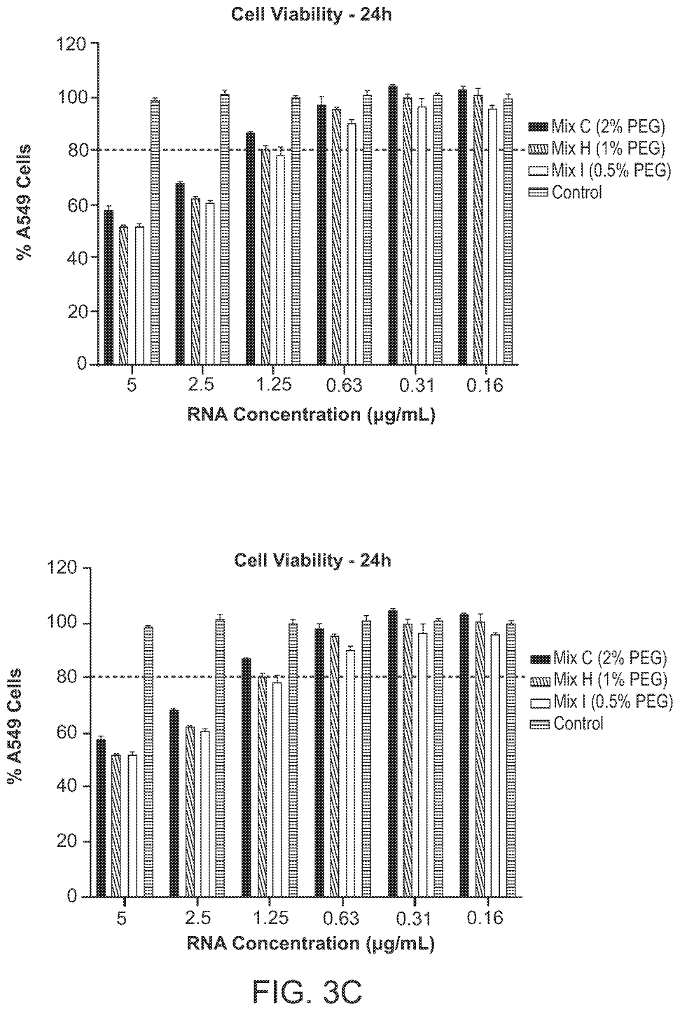

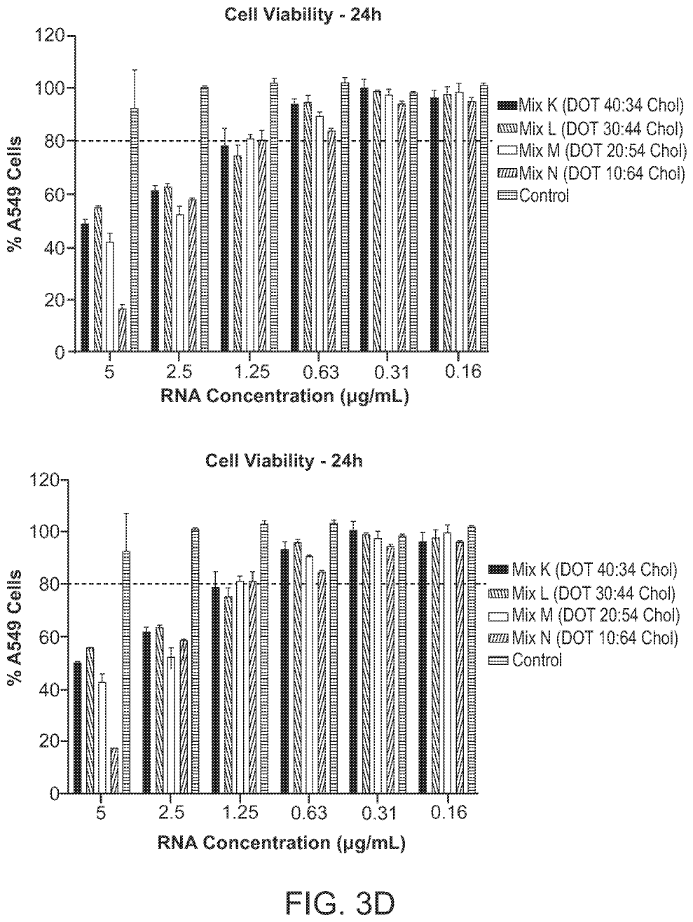

In some embodiments of the instant disclosure, DOTAP/C12-200-based LNPs were prepared using a microfluidic mixing process. Briefly, lipid stocks of DOTAP, C12-200, DOPE, CHE and PEG-DMG were prepared in ethanol at 20 mg/ml concentration (it is noted that lipid stocks of 20 mg/ml to 80 mg/ml can readily be used, e.g., for animal studies). Different PEGylation levels (0-2%) and lipid compositions (molar ratio between the lipids to each other) were prepared and assessed. In all formulations, the DOTAP mol percent was varied between 10-50, with variations in DOTAP levels offset by altering DOPE and/or cholesterol levels, while the C12-200 mol percent was held at 18. Lipids were mixed together for the given compositions in ethanol with a final lipid concentration of 6.5-8.5 mg/mL for in vitro studies, or of 15-120 mg/mL for animal studies. mCherry protein-encoding mRNA (mCherry) was used as the mRNA in the aqueous phase at a concentration of 0.25-2 mg/ml. The mixing of two phases and LNP preparation was performed using a 2:1 or 3:1 aqueous to organic volume ratio, and at an 8 or 12 ml/min flow rate in a microfluidic chip with staggered herringbone structure. Resulting LNPs were subjected to purification and buffer exchange by tangential flow filtration (TFF) against molecular biology grade water. Alternatively, resulting LNPs were subjected to dialysis against molecular biology grade water using a membrane with a MWCO range between 8-300 kDa. Characterization parameters of the formulations are summarized below and in FIG. 3A. Precise control of the characterization parameters enabled the preparation of DOTAP/C12-200-based LNPs in the size range of 70-100 nm, surface charge (Zeta values) between 3-16 mV, and PDI below 0.22.

Lipid particles prepared according to methods as disclosed herein and as known in the art can in certain embodiments be stored for substantial periods of time prior to drug loading and administration to a patient. For example, lipid nanoparticles can be dehydrated, stored, and subsequently rehydrated and loaded with one or more active agents, prior to administration. Lipid nanoparticles may also be dehydrated after being loaded with one or more active agents. Dehydration can be accomplished by a variety of methods available in the art, including the dehydration and lyophilization procedures described, e.g., in U.S. Pat. Nos. 4,880,635, 5,578,320, 5,837,279, 5,922,350, 4,857,319, 5,376,380, 5,817,334, 6,355,267, and 6,475,517. In one embodiment, lipid nanoparticles are dehydrated using standard freeze-drying apparatus, i.e., they are dehydrated under low pressure conditions. Also, the lipid nanoparticles can be frozen, e.g., in liquid nitrogen, prior to dehydration. Sugars can be added to the LNP environment, e.g., to the buffer containing the lipid nanoparticles, prior to dehydration, thereby promoting the integrity of the lipid nanoparticle during dehydration. See, e.g., U.S. Pat. No. 5,077,056 or 5,736,155.

Lipid nanoparticles may be sterilized by conventional methods at any point during their preparation, including, e.g., after sizing or after generating a pH gradient.

### Cargo-Loaded Lipid Particle Compositions

In various embodiments, lipid particles of the instant disclosure may be used for many different applications, including the delivery of an active agent to a cell, tissue, organ or subject. For example, lipid nanoparticles of the instant disclosure may be used to deliver a therapeutic agent systemically via the bloodstream or to deliver a cosmetic agent to the skin. Accordingly, lipid nanoparticles of the instant disclosure and one or more active agents as cargo(es) are included in the instant disclosure.

### Lipid Particle Cargoes

The instant disclosure describes mixed cationic lipid nanoparticles (i.e., a lipid nanoparticle comprising an ionizable lipid (e.g., C12-200) and another cationic lipid (e.g., DOTAP)) in combination with an active agent as a cargo. Active agents, as used herein, include any molecule or compound capable of exerting a desired effect on a cell, tissue, organ, or subject. Such effects may be biological, physiological, or cosmetic, for example. Active agents may be any type of molecule or compound, including e.g., nucleic acids, such as single- or double-stranded polynucleotides, plasmids, antisense RNA, RNA interference agents, including, e.g., DNA-DNA hybrids, DNA-RNA hybrids, RNA-DNA hybrids, RNA-RNA hybrids, short interfering RNAs (siRNA), micro RNAs (mRNA) and short hairpin RNAs (shRNAs); peptides and polypeptides, including, e.g., antibodies, such as, e.g., polyclonal antibodies, monoclonal antibodies, antibody fragments; humanized antibodies, recombinant antibodies, recombinant human antibodies, and Primatized™ antibodies, cytokines, growth factors, apoptotic factors, differentiation-inducing factors, cell surface receptors and their ligands; hormones; and small molecules, including small organic molecules or compounds.

Nucleic acids associated with or encapsulated by LNPs may contain modifications including but not limited to those selected from the following group: 2′-O-methyl modified nucleotides, a nucleotide comprising a 5′-phosphorothioate group, a terminal nucleotide linked to a cholesteryl derivative, a 2′-deoxy-2′-fluoro modified nucleotide, a 5′-methoxy-modified nucleotide (e.g., 5′-methoxyuridine), a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide; internucleoside linkages or backbones including phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2.′

In certain embodiments, the active agent is a mRNA or a vector capable expressing a mRNA in a cell.

In embodiments, the active agent is a CRISPR/Cas system. Optionally, a LNP of the instant disclosure can be formulated to include, e.g., both a guide strand (gRNA) and a Cas enzyme as cargoes, thereby providing a self-contained delivery vehicle capable of effecting and controlling CRISPR-mediated targeting of a gene in a target cell.

In certain featured embodiments, the active agent is a nucleic acid modulating controller (e.g., a mRNA that encodes protein controller components, as described above).

In some embodiments, the active agent is a therapeutic agent, or a salt or derivative thereof. Therapeutic agent derivatives may be therapeutically active themselves or they may be prodrugs, which become active upon further modification. Thus, in one embodiment, a therapeutic agent derivative retains some or all of the therapeutic activity as compared to the unmodified agent, while in another embodiment, a therapeutic agent derivative lacks therapeutic activity.

In various embodiments, therapeutic agents include agents and drugs, such as anti-inflammatory compounds, narcotics, depressants, anti-depressants, stimulants, hallucinogens, analgesics, antibiotics, birth control medication, antipyretics, vasodilators, anti-angiogenics, cytovascular agents, signal transduction inhibitors, vasoconstrictors, hormones, and steroids.

In certain embodiments, the active agent is an oncology drug, which may also be referred to as an anti-tumor drug, an anti-cancer drug, a tumor drug, an antineoplastic agent, or the like. Examples of oncology drugs that may be used according to the instant disclosure include, but are not limited to, adriamycin, alkeran, allopurinol, altretamine, amifostine, anastrozole, araC, arsenic trioxide, azathioprine, bexarotene, biCNU, bleomycin, busulfan intravenous, busulfan oral, capecitabine (Xeloda), carboplatin, carmustine, CCNU, celecoxib, chlorambucil, cisplatin, cladribine, cyclosporin A, cytarabine, cytosine arabinoside, daunorubicin, cytoxan, daunorubicin, dexamethasone, dexrazoxane, dodetaxel, doxorubicin, doxorubicin, DTIC, epirubicin, estramustine, etoposide phosphate, etoposide and VP-16, exemestane, FK506, fludarabine, fluorouracil, 5-FU, gemcitabine (Gemzar), gemtuzumab-ozogamicin, goserelin acetate, hydrea, hydroxyurea, idarubicin, ifosfamide, imatinib mesylate, interferon, irinotecan (Camptostar, CPT-111), letrozole, leucovorin, leustatin, leuprolide, levamisole, litretinoin, megastrol, melphalan, L-PAM, mesna, methotrexate, methoxsalen, mithramycin, mitomycin, mitoxantrone, nitrogen mustard, paclitaxel, pamidronate, Pegademase, pentostatin, porfimer sodium, prednisone, rituxan, streptozocin, STI-571, tamoxifen, taxotere, temozolamide, teniposide, VM-26, topotecan (Hycamtin), toremifene, tretinoin, ATRA, valrubicin, velban, vinblastine, vincristine, VP16, and vinorelbine. Other examples of oncology drugs that may be used according to the instant disclosure are ellipticin and ellipticin analogs or derivatives, epothilones, intracellular kinase inhibitors and camptothecins.

While LNP compositions of the instant disclosure generally comprise a single active agent, in certain embodiments, they may comprise more than one active agent.

In other embodiments of the instant disclosure, the lipid nanoparticles of the instant disclosure have a plasma circulation half-life of at least 0.5, 0.8, 1.2, 1.5, 2.0, 4.0, 6.0, 8.0, or 12 hours. In some embodiments, lipid nanoparticles have a plasma drug half-life of at least 0.5, 0.8, 1.2, 1.5, 2.0, 4.0, 6.0, 8.0, or 12 hours. Circulation and blood or plasma clearance half-lives may be determined as described, for example, in U.S. Patent Publication No. 2004-0071768-A1.

The instant disclosure also provides lipid nanoparticles and variations thereof in kit form. The kit may comprise a ready-made formulation or a formulation that requires mixing before administration. The kit will typically comprise a container that is compartmentalized for holding the various elements of the kit. The kit will contain the lipid nanoparticle compositions of the instant disclosure or the components thereof, in hydrated or dehydrated form, with instructions for their rehydration and administration. In particular embodiments, a kit comprises at least one compartment containing a lipid nanoparticle of the instant disclosure that is loaded with an active agent. In another embodiment, a kit comprises at least two compartments, one containing a lipid nanoparticle of the instant disclosure and the other containing an active agent. Of course, it is understood that any of these kits may comprise additional compartments, e.g., a compartment comprising a buffer, such as those described in U.S. Patent Publication No. 2004-0228909-A1. Kits of the instant disclosure, which comprise lipid nanoparticles comprising mixed cationic lipids (e.g., ionizable lipid such as C12-200 and other cationic lipid such as DOTAP), may also contain other features of the kits described in U.S. Patent Publication No. 2004-0228909 A1. Further, the kit may contain drug-loaded lipid nanoparticles in one compartment and empty lipid nanoparticles in a second compartment. Alternatively, the kit may contain a lipid nanoparticle of the instant disclosure, an active agent to be loaded into the lipid nanoparticle of the instant disclosure in a second compartment, and an empty lipid nanoparticle in a third compartment.

In a particular embodiment, a kit of the instant disclosure comprises a therapeutic compound encapsulated in a mixed lipid nanoparticle comprising both C12-200 and DOTAP, where C12-200 constitutes 5-20% (molar basis) of total lipids present in the lipid nanoparticle and DOTAP comprises 10-50% (molar basis) of total lipids present in the lipid nanoparticle, as well as an empty lipid nanoparticle. In one embodiment, the lipid nanoparticle containing therapeutic compound and the empty lipid nanoparticle are present in different compartments of the kit.

### Efficacy of Lipid Particle-Mediated Cargo Delivery

In certain embodiments, the instant disclosure is based, at least in part, upon the surprising result that particles containing from 10-50% (mol/weight) DOTAP mixed with 18% C12-200 lipid particles are highly effective at delivering active nucleic acid cargoes (including even large nucleic acid regulatory controllers) into cells of the liver, relative to other tissues. Further, reporter activity of the encapsulated active agent (cargo), e.g. mRNA, occurred almost exclusively in the liver tissue, as compared to other tissues, and nuclear localization and efficacy of such cargoes was also demonstrated. The efficacy of localization of a lipid particle may be described as the fold difference (increase or decrease) in localization of the nucleic acid-lipid particle to a particular tissue of the subject relative to that of one or more other tissues of the subject. The efficacy of activity as a further component in assessing delivery may be described as the fold difference (increase or decrease) in activity of the active agent, e.g., a nucleic acid cargo or other compound, within cells of a particular tissue of the subject, relative to that observed in cells of one or more other tissues of the subject. In some embodiments, the fold difference may therefore be detected at the cellular level, or can be detected by appropriate proxy for events occurring at the cellular level. In some embodiments, the cell of the liver tissue affected is one or more of a hepatocyte, a vascular cell (i.e. hepatic sinusoid), a hepatic stellate cell, an endothelial cell, a fibroblast, a mesenchymal cell, an immune cell, a cancer cell, a Kupffer cell, an astrocyte, an oval-shaped vascular endothelial cell, a liver-derived stem/progenitor cell, and a stem/progenitor or cancer cell derived from non-liver tissue. In some embodiments, the fold-difference in effect/activity may be detected at a sub-cellular level, i.e. where activity is detectible in the nuclei of targeted cells.

To determine the efficacy of localization of the LNP, assays may be performed according to the characteristics of the labeled or detected molecule of interest. In illustrative embodiments of the instant disclosure, a fluorescently labeled lipid has been used to determine LNP localization. In other embodiments, a labeled peptide, or other component of a lipid particle may be used. In some embodiments, the localization is detectible in individual cells. In some embodiments the label is a fluorescent label, i.e. a fluorescently labeled lipid such as Cy7. In other embodiments the label of the lipid nanoparticle may be a quantum dot, or the lipid detectible by stimulated Raman scattering. In other embodiments the label is any fluorophore known in the art, i.e. with excitation and emission in the ultraviolet, visible, or infrared spectra. In some embodiments the localization is detected or further corroborated by immunohistochemistry or immunofluorescence methods.

The efficacy of localization may be described as the fold difference (increase or decrease) in localization of the nucleic acid-lipid particle to a tissue, i.e. liver tissue, of the subject relative to one or more other tissues of the subject. In illustrative embodiments of the instant disclosure, Cy7 labeled lipids were imaged in vivo and fluorescence radiance served as an indication of Cy7-LNP concentration (see Example 6 below). The Cy7-DOPE-labeled DOTAP/C12-200 nucleic acid LNPs exhibited increased efficacy of localization to the liver relative to other tissues, in particular relative to the lung, heart, and spleen. In some embodiments of the instant disclosure a Cy7 labeled nucleic acid LNP, exhibited at least two-fold localization to the liver relative to the lungs, heart, or spleen. In some embodiments, a Cy7 labeled nucleic acid LNP, exhibited at least three-fold localization to the liver, in some embodiments a Cy7 labeled nucleic acid LNP exhibited at least four-fold localization to the liver, in some embodiments the Cy7 labeled nucleic acid LNP exhibited at least five-fold localization to the liver, in some embodiments the Cy7 labeled a nucleic acid LNP exhibited at least six-fold localization to the liver, in some embodiments the Cy7 labeled a nucleic acid LNP exhibited at least ten-fold localization to the liver, in some embodiments the Cy7 labeled a nucleic acid LNP exhibited at least fifteen-fold localization to the liver, in some embodiments the Cy7 labeled a nucleic acid LNP exhibited at least twenty-fold localization to the liver, relative to that of the lung, heart, and spleen.

To determine the efficacy of activity of the active agent encapsulated by the lipid particle, assays may be performed according to the characteristics of the active agent. In certain embodiments, the active agent in the mixed lipid particle is a nucleic acid. In other embodiments, the active agent in the mixed lipid particle is a small molecule or other compound.

In some embodiments, the active agent in the mixed lipid particle is a mRNA. In illustrative embodiments, the localized expression of a reporter mRNA, i.e. mCherry, served as an indication of intracellular delivery efficacy for an mRNA as the active agent/cargo. In other embodiments, the mRNA may encode Cre enzyme (as used in confirming nuclear delivery and activity of cargo mRNA via the tdTomato reporter system of Example 6 herein), green fluorescent protein, red fluorescent protein, yellow fluorescent protein or blue fluorescent protein. Or in therapeutic embodiments, the mRNA may encode for a protein for therapeutic intracellular expression in LNP-targeted cells of a subject (including delivery and expression of nucleic acid modulatory controllers), optionally where intracellular levels of delivered mRNA or encoded protein can be detected by methods known in the art, as appropriate for the therapeutic mRNA that is delivered. In other embodiments, a reporter mRNA encodes a cell surface marker, such as a Lyt2 cell surface marker. In still other embodiments, the reporter can be a β-galactosidase, α-lactamase, an alkaline phosphatase or a horse-radish peroxidase. In other embodiments, the reporter mRNA encodes a negative selection marker, such as thymidine kinase (tk), HRPT or APRT. In some embodiments, immunohistochemistry or immunofluorescence is used to detect or corroborate activity of the reporter mRNA.

In certain embodiments, the effectiveness of a lipid particle of the instant disclosure in delivering a cargo is assessed based upon the levels of activity observed for the cargo (active agent) intracellularly within a lipid particle-targeted tissue. Such effects can be identified as fold-differences in activity, as compared to an appropriate control formulation and/or tissue, e.g., the delivery efficacy of a LNP with nucleic acid cargo may be described as the fold difference (increase or decrease) in activity of the nucleic acid cargo in cells of a targeted tissue, i.e. the liver tissue, of a subject relative to one or more other tissues of the subject. Thus, for certain nucleic acid cargoes, delivery efficacy of an LNP formulation can be identified as a LNP that achieves, e.g., two-fold greater intracellular activity of the nucleic acid payload in targeted tissue cells than in non-targeted tissue cells, or relative to a LNP formulation that does not include the nucleic acid cargo. Optionally, an effective LNP formulation for delivery of a nucleic acid cargo can be described as one that achieves at least about a three-fold greater, optionally about a four-fold greater, optionally about a five-fold greater, optionally about a six-fold greater, optionally about a seven-fold greater, optionally about an eight-fold greater, optionally about a nine-fold greater, optionally about a ten-fold greater, optionally about a twenty-fold greater, optionally about a 50-fold greater, optionally about a 100-fold greater, etc. intracellular activity of the nucleic acid payload in targeted tissue cells than in non-targeted tissue cells, or relative to a LNP formulation that does not include the nucleic acid cargo. In illustrative embodiments of the instant disclosure, mixed lipid particles delivered a mCre mRNA that was expressed in cells of the liver tissue of the subject at a level that was significantly higher than that of cells of the lungs, heart, and spleen of the subject (see Example 6 below). Cells expressing mCre were detected through imaging of a Cre expression-responsive tdTomato reporter. In some embodiments, the Cre mRNA was expressed in cells of the liver tissue of the subject at a level that was at least two-fold higher than expression of the mRNA in cells of the lung, heart, and spleen of the subject. In some embodiments, cargo mRNA was expressed in cells of the liver tissue of the subject at a level that was at least three-fold the higher than expression of the mRNA in cells of the lungs, heart, and spleen of the subject. In some embodiments, the luciferase mRNA was expressed at least four-fold higher in the liver, in some embodiments, the luciferase mRNA was expressed at least five-fold higher in the liver, in some embodiments at least six-fold higher in the liver, in some embodiments at least seven-fold higher in the liver, in some embodiments at least eight-fold higher in the liver, in some embodiments at least nine-fold higher in the liver, in some embodiments at least ten-fold higher in the liver, in some embodiments at least eleven-fold higher in the liver, in some embodiments at least twelve-fold higher in the liver, in some embodiments at least thirteen-fold higher in the liver, in some embodiments at least fourteen-fold higher in the liver, in some embodiments at least fifteen-fold higher in the liver, in some embodiments at least twenty-fold higher in the liver, than expression of the cargo mRNA in cells of the lungs, heart, and spleen of the subject.

In other embodiments, mixed lipid particles can be employed to deliver a RNAi agent (e.g., a siRNA) to a tissue, i.e. a liver tissue. For siRNA or other RNAi agents, delivery and activity efficacy measurements can employ, for example, target-specific PCR to detect transcript levels, immunosorbent or other immunological methods to detect target protein levels, and/or Flow Cytometry (FACS) (Testoni et al., Blood 1996, 87:3822). In some embodiments, a siRNA may be active in cells of the liver tissue of the subject at a level that is at least two-fold higher than in cells of the lungs, heart, spleen, ovary, pancreas, kidney and/or other non-liver organ or tissue of the subject. In some embodiments, a siRNA may be active in cells of the liver tissue of the subject at a level that is at least three-fold higher than in cells of the lungs, heart, spleen, ovary, pancreas, kidney and/or other non-liver organ or tissue of the subject. In some embodiments, the siRNA may be active at a level at least four-fold higher in the liver, in some embodiments, the siRNA may be active at a level at least five-fold higher in the liver, in some embodiments the siRNA may be active at a level at least six-fold higher in the liver, in some embodiments at least seven-fold higher in the liver, in some embodiments at least eight-fold higher in the liver, in some embodiments at least nine-fold higher in the liver, in some embodiments at least ten-fold higher in the liver, in some embodiments at least eleven-fold higher in the liver, in some embodiments at least twelve-fold higher in the liver, in some embodiments at least thirteen-fold higher in the liver, in some embodiments at least fourteen-fold higher in the liver, in some embodiments at least fifteen-fold higher in the liver, in some embodiments at least twenty-fold higher in the liver, etc., than activity of the siRNA in cells of the lungs, heart, spleen, ovary, pancreas, kidney and/or other non-liver organ or tissue of the subject. In related embodiments, a mixed lipid particle that delivers a RNAi cargo preferentially to the liver may exhibit, e.g., greater than 20% reduction in target transcript and/or protein levels in cells of targeted liver tissue, as compared to cells of non-targeted tissues or as compared to some other appropriate control (e.g., levels of target transcript in untreated liver tissue cells). Optionally, a mixed lipid particle that delivers a RNAi cargo preferentially to the liver may exhibit, e.g., more than 30% reduction, more than 40% reduction, more than 50% reduction, more than 60% reduction, more than 70% reduction, more than 80% reduction, more than 90% reduction, more than 95% reduction, more than 97% reduction, more than 97% reduction, more than 98% reduction or more than 99% reduction in target transcript and/or protein levels in cells of targeted liver tissue, as compared to cells of non-targeted tissues or as compared to some other appropriate control (e.g., levels of target transcript in untreated liver tissue cells).

In some embodiments, mixed lipid particles of the instant disclosure can be used to deliver a CRISPR-Cas9 system to a tissue, i.e. a liver tissue. CRISPR-Cas9 delivery and activity efficacy measurements may require, for example, PCR to detect Cas9, the genomic structures of targeted regions and/or target transcript levels, immunosorbent or other immunological methods to detect Cas9 or knock-in, knock-out, or other modifications of target proteins, and/or Flow Cytometry (FACS) (Testoni et al., Blood 1996, 87:3822). In some embodiments, CRISPR-Cas9-mediated effects may be identified in cells of the liver tissue of the subject at a level that is at least two-fold higher than in cells of the lungs, heart, spleen, ovary, pancreas, kidney and/or other non-liver organ or tissue of the subject. In some embodiments, CRISPR-Cas9-mediated effects may be identified in cells of the liver tissue of the subject at a level that is at least three-fold higher than in cells of the lungs, heart, spleen, ovary, pancreas, kidney and/or other non-liver organ or tissue of the subject. In some embodiments, CRISPR-Cas9-mediated effects may be identified in cells at a level at least four-fold higher in the liver, in some embodiments, CRISPR-Cas9-mediated effects may be identified in cells at a level at least five-fold higher in the liver, in some embodiments, CRISPR-Cas9-mediated effects may be identified in cells at a level at least six-fold higher, in some embodiments, CRISPR-Cas9-mediated effects may be identified in cells at least seven-fold higher, in some embodiments at least eight-fold higher, in some embodiments at least nine-fold higher, in some embodiments at least ten-fold higher, in some embodiments at least eleven-fold higher, in some embodiments at least twelve-fold higher, in some embodiments at least thirteen-fold higher, in some embodiments at least fourteen-fold higher, in some embodiments at least fifteen-fold higher, in some embodiments at least twenty-fold higher in liver, than CRISPR-Cas9-mediated effects in cells of the lungs, heart, spleen, ovary, pancreas, kidney and/or other non-liver organ or tissue of the subject.

In other embodiments, mixed lipid particles may deliver a mRNA or other nucleic acid cargo to a tissue, i.e. a liver tissue, where expression and possibly activity occurs in the nucleus. In the instant disclosure, some embodiments have utilized the Cre recombinase enzyme as a reporter for nuclear activity of the active agent (see Example 6 below). The Cre recombinase enzyme requires translocation of the encoded protein to the nucleus and thus can serve as a reporter of nuclear translocation. The Cre recombinase catalyzes site-specific recombination of DNA between loxP sites. Upon Cre recombinase activity expression, due to loxP recombination, reporter fluorescent proteins are expressed. In one embodiment, the Ai14 mouse line used a Cre reporter loxP-flanked STOP cassette preventing transcription of a CAG promoter-driven red fluorescent protein variant (tdTomato), inserted into the Gt(ROSA)26Sor locus. The Ai14 mice were intravenously injected with mCre-loaded DOTAP-LNPs and began expressing robust tdTomato fluorescence in the nuclei of liver cells following delivery and expression of the Cre enzyme, nuclear translocation of the Cre enzyme, and subsequently, Cre-mediated recombination of the tdTomato promoter. In exemplary embodiments, the efficacy of activity, i.e. the expression of a mRNA detectible in the nucleus, was observable in the nucleus of liver cells at a level at least two-fold higher than that of cells in the lung, heart, and spleen. In some embodiments, the expression of a mRNA detectible in the nucleus was observable in the nucleus of liver cells at a level at least three-fold higher than that of cells in the lung, heart, and spleen. In some embodiments, the expression of an mRNA detectible in the nucleus was observable in the nucleus of liver cells at a level at least four-fold higher, in some embodiments the level was five-hold higher, in some embodiments, six-fold higher, in some embodiments, seven-fold higher, in some embodiments, eight-fold higher, in some embodiments, nine-fold higher, in some embodiments, ten-fold higher, in some embodiments, eleven-fold higher, in some embodiments, twelve-fold higher, in some embodiments, thirteen-fold higher, in some embodiments, fourteen-fold higher, in some embodiments, fifteen-fold higher, in some embodiments, twenty-fold higher, than activity of the mRNA detectible in the nucleus in cells of the lung, heart, and spleen.

In other embodiments, mixed lipid particles may deliver small molecules or other compounds to a tissue, i.e. a liver tissue. The efficacy of localization or activity of small molecules may be determined by a number of in vivo imaging methods (e.g. PET/CT), mass spectrometry, as well as immunohistochemistry and immunofluorescence of target effects. In some embodiments, the mixed lipid particle-mediated localization and/or activity of the small molecule in the liver may be two-fold higher than that of other tissues, for example than that of the lungs, heart, kidney, ovary, pancreas or other tissues, optionally than that of the spleen. In some embodiments, the mixed lipid particle-mediated localization and/or activity of the small molecule in the liver may be three-fold higher than that of other tissues, four-fold higher, five-hold higher, six-fold higher, seven-fold higher, eight-fold higher, nine-fold higher, ten-fold higher, eleven-fold higher, twelve-fold higher, thirteen-fold higher, fourteen-fold higher, fifteen-fold higher, or twenty-fold higher than that of other tissues, for example than that of the lungs, heart, kidney, ovary, pancreas or other tissues, optionally than that of the spleen.

In certain embodiments, a lipid particle that is formulated for liver delivery refers to a lipid particle that exhibits preferential localization and intracellular delivery (based upon assessment of intracellular activity either directly or by proxy) of a cargo to liver cells, as compared to cells of one or more other tissues of a subject. For example, a lipid particle for liver delivery is one capable of inducing at least two-fold greater activity of a cargo (e.g., a nucleic acid cargo, e.g., a mRNA, a CRISPR/Cas system, a nucleic acid modulating controller, etc.) in liver cells of a subject, than in other tissues of the subject. Such effects in liver cells of a subject can be evaluated within one or more cell types of the liver, as described elsewhere herein. In certain embodiments, a lipid particle for liver delivery is one capable of inducing at least three-fold greater, at least four-fold greater, at least five-fold greater, at least six-fold greater, at least seven-fold greater, at least eight-fold greater, at least nine-fold greater, at least ten-fold greater, at least fifteen-fold greater, at least twenty-fold greater, at least thirty-fold greater, at least 40-fold greater, at least 50-fold greater, at least 60-fold greater, at least 70-fold greater, at least 80-fold greater, at least 90-fold greater, at least 100-fold greater, at least 1000-fold greater, etc. activity of a cargo (e.g., a nucleic acid cargo, e.g., a mRNA, a CRISPR/Cas system, a nucleic acid modulating controller, etc.) in liver cells of a subject, than in other tissues of the subject.

In still other embodiments, mixed lipid particles formulated without PEG modified lipids may diminish or avoid the accelerated blood clearance effect (ABC), where the immune system targets PEG for removal.

### LNP-Mediated Cargo Delivery

The lipid particle compositions disclosed herein can be used for a variety of purposes, including the delivery of an active agent or therapeutic agent or compound to a subject or patient in need thereof. Subjects include both humans and non-human animals. In certain embodiments, subjects are mammals. In other embodiments, subjects are one or more particular species or breed, including, e.g., humans, mice, rats, dogs, cats, cows, pigs, sheep, or birds.

Thus, the instant disclosure also provides methods of treatment for a variety of diseases and disorders, as well as methods intended to provide a cosmetic benefit.

### Methods of Treatment

The LNP compositions of the instant disclosure may be used to treat any of a wide variety of diseases or disorders, including, but not limited to, inflammatory diseases, cardiovascular diseases, nervous system diseases, tumors, demyelinating diseases, digestive system diseases, endocrine system diseases, reproductive system diseases, hemic and lymphatic diseases, immunological diseases, mental disorders, musculoskeletal diseases, neurological diseases, neuromuscular diseases, metabolic diseases, sexually transmitted diseases, skin and connective tissue diseases, urological diseases, and infections.

In certain embodiments, the LNP compositions can be employed to treat or prevent a liver disease or disorder, including but not limited to a disease or disorder selected from the following: biliary atresia, Alagille Syndrome, alpha-1 antitrypsin deficiency, Tyrosinemia, neonatal hepatitis, hepatitis C virus infection, hepatitis B virus infection, hepatitis A virus infection, hepatocellular carcinoma and Wilson's disease.

In other embodiments, the LNP compositions of the instant disclosure can be used to treat or prevent a joint disease or disorder, including but not limited to a disease or disorder selected from the following: rheumatoid arthritis, psoriatic arthritis, gout, tendinitis, bursitis, Carpal Tunnel Syndrome, and osteoarthritis.

In other embodiments, the LNP compositions of the instant disclosure can be used to treat or prevent an inflammatory disease or disorder, including but not limited to a disease or disorder selected from the following: inflammatory bowel disease, peritonitis, osteomyelitis, cachexia, pancreatitis, trauma induced shock, bronchial asthma, allergic rhinitis, cystic fibrosis, acute bronchitis, acute intense bronchitis, osteoarthritis, rheumatoid arthritis, infectious arthritis, post-infectious arthritis, gonocoele arthritis, tuberculous arthritis, arthritis, osteoarthritis, gout, spondyloarthropathies, ankylosing spondylitis, arthritis associated with vasculitis syndrome, nodular polyarteritis nervosa, irritable vasculitis, rugenic granulomatosis, rheumatoid polyposis myalgia, arthritis cell arteritis, calcium polycystic arthropathy, caustic gout, non-arthritic rheumatism, bursitis, hay fever, suppurative inflammation (e.g., tennis elbow), neuropathic joint disease, hemarthrosic, Henoch-Schlein purpura, hypertrophic osteoarthritis, multisized hemorrhoids, scoliosis, hemochromatosis, hyperlipoproteinemia, hypogammaglobulinemia, COPD, acute respiratory distress syndrome, acute lung injury, broncho-pulmonary dysplasia and systemic lupus erythematosus (SLE).

In other embodiments, the LNP compositions of the instant disclosure can be used to treat or prevent an epidermal disease or disorder, including but not limited to psoriasis, atopic dermatitis, scleroderma, eczema, rosacea, seborrheic dermatitis, melanoma, solar keratosis, ichthyosis, Grover's disease, common warts, keratoacanthoma, and seborrhoeic keratosis.

In one embodiment, the LNP compositions of the instant disclosure can be used to treat or prevent a type of cancer. In particular, these methods can be applied to cancers of the blood and lymphatic systems, including lymphomas, leukemia, and myelomas. Examples of specific cancers that may be treated according to the instant disclosure include, but are not limited to, Hodgkin's and non-Hodgkin's Lymphoma (NHL), including any type of NHL as defined according to any of the various classification systems such as the Working formulation, the Rappaport classification and, preferably, the REAL classification. Such lymphomas include, but are not limited to, low-grade, intermediate-grade, and high-grade lymphomas, as well as both B-cell and T-cell lymphomas. Included in these categories are the various types of small cell, large cell, cleaved cell, lymphocytic, follicular, diffuse, Burkitt's, Mantle cell, NK cell, CNS, AIDS-related, lymphoblastic, adult lymphoblastic, indolent, aggressive, transformed and other types of lymphomas. The methods of the instant disclosure can be used for adult or childhood forms of lymphoma, as well as lymphomas at any stage, e.g., stage I, II, III, or IV. The various types of lymphomas are well known to those of skill, and are described, e.g., by the American Cancer Society (see, e.g., www3.cancer.org).

The compositions and methods described herein may also be applied to any form of leukemia, including adult and childhood forms of the disease. For example, any acute, chronic, myelogenous, and lymphocytic form of the disease can be treated using the methods of the instant disclosure. In preferred embodiments, the methods are used to treat Acute Lymphocytic Leukemia (ALL). More information about the various types of leukemia can be found, inter alia, from the Leukemia Society of America (see, e.g., www.leukemia.org).

Additional types of tumors can also be treated using the methods described herein, such as neuroblastomas, myelomas, prostate cancers, small cell lung cancer, colon cancer, ovarian cancer, non-small cell lung cancer, brain tumors, breast cancer, and others.

The LNP compositions of the instant disclosure may be administered as first line treatments or as secondary treatments. In addition, they may be administered as a primary chemotherapeutic treatment or as adjuvant or neoadjuvant chemotherapy. For example, treatments of relapsed, indolent, transformed, and aggressive forms of non-Hodgkin's Lymphoma may be administered following at least one course of a primary anti-cancer treatment, such as chemotherapy and/or radiation therapy.

### Administration of LNP Compositions

LNP compositions of the instant disclosure are administered in any of a number of ways, including parenteral, intravenous, systemic, local, oral, intratumoral, intramuscular, subcutaneous, intraperitoneal, inhalation, or any such method of delivery. In one embodiment, the compositions are administered parenterally, i.e., intraarticularly, intravenously, intraperitoneally, subcutaneously, or intramuscularly. In a specific embodiment, the LNP compositions are administered by intravenous infusion or intraperitoneally by a bolus injection. For example, in one embodiment, a patient is given an intravenous infusion of the lipid nanoparticle-encapsulated active agent through a running intravenous line over, e.g., 5-10 minutes, 15-20 minutes, 30 minutes, 60 minutes, 90 minutes, or longer. In one embodiment, a 60 minute infusion is used. In other embodiments, an infusion ranging from 6-10 or 15-20 minutes is used. Such infusions can be given periodically, e.g., once every 1, 3, 5, 7, 10, 14, 21, or 28 days or longer, preferably once every 7-21 days, and preferably once every 7 or 14 days.

LNP compositions of the instant disclosure may be formulated as pharmaceutical compositions suitable for delivery to a subject. The pharmaceutical compositions of the instant disclosure will often further comprise one or more buffers (e.g., neutral buffered saline or phosphate buffered saline), carbohydrates (e.g., glucose, mannose, sucrose, dextrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the instant disclosure may be formulated as a lyophilizate.

The concentration of drug and lipid nanoparticles in the pharmaceutical formulations can vary widely, i.e., from less than about 0.05%, usually at or at least about 2-5% to as much as 10 to 30% by weight and will be selected depend upon the particular drug used, the disease state being treated and the judgment of the clinician taking. Further, the concentration of drug and lipid nanoparticles will also take into consideration the fluid volume administered, the osmolality of the administered solution, and the tolerability of the drug and lipid nanoparticles. In some instances, it may be preferable to use a lower drug or lipid nanoparticle concentration to reduce the incidence or severity of infusion-related side effects.

Suitable formulations for use in the instant disclosure can be found, e.g., in Remington's Pharmaceutical Sciences, Mack Publishing Company, Philadelphia, Pa., 17^{th} Ed. (1985). Often, intravenous compositions will comprise a solution of the lipid nanoparticles suspended in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and may include glycoproteins for enhanced stability, such as albumin, lipoprotein, globulin, etc. Often, normal buffered saline (135-150 mM NaCl) or 5% dextrose will be used. These compositions can be sterilized by conventional sterilization techniques, such as filtration. The resulting aqueous solutions may be packaged for use or filtered under aseptic conditions and lyophilized, the lyophilized preparation being combined with a sterile aqueous solution prior to administration. The compositions may also contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. Additionally, the composition may include lipid-protective agents, which protect lipids against free-radical and lipid-peroxidative damages on storage. Lipophilic free-radical quenchers, such as α-tocopherol and water-soluble iron-specific chelators, such as ferrioxamine, are suitable.

The amount of active agent administered per dose is selected to be above the minimal therapeutic dose but below a toxic dose. The choice of amount per dose will depend on a number of factors, such as the medical history of the patient, the use of other therapies, and the nature of the disease. In addition, the amount of active agent administered may be adjusted throughout treatment, depending on the patient's response to treatment and the presence or severity of any treatment-associated side effects. In certain embodiments, the dosage of LNP composition or the frequency of administration is approximately the same as the dosage and schedule of treatment with the corresponding free active agent. However, it is understood that the dosage may be higher or more frequently administered as compared to free drug treatment, particularly where the LNP composition exhibits reduced toxicity. It is also understood that the dosage may be lower or less frequently administered as compared to free drug treatment, particularly where the LNP composition exhibits increased efficacy as compared to the free drug. Exemplary dosages and treatment for a variety of chemotherapy compounds (free drug) are known and available to those skilled in the art and are described in, e.g., Physician's Cancer Chemotherapy Drug Manual, E. Chu and V. Devita (Jones and Bartlett, 2002).

Patients typically will receive at least two courses of such treatment, and potentially more, depending on the response of the patient to the treatment. In single agent regimens, total courses of treatment are determined by the patient and physician based on observed responses and toxicity.

### Combination Therapies

In certain embodiments, LNP compositions of the instant disclosure can be administered in combination with one or more additional compounds or therapies, such as surgery, radiation treatment, chemotherapy, or other active agents, including any of those described above. LNP compositions may be administered in combination with a second active agent for a variety of reasons, including increased efficacy or to reduce undesirable side effects. The LNP composition may be administered prior to, subsequent to, or simultaneously with the additional treatment. Furthermore, where a LNP composition of the instant disclosure (which comprises a first active agent) is administered in combination with a second active agent, the second active agent may be administered as a free drug, as an independent LNP formulation, or as a component of the LNP composition comprising the first drug. In certain embodiments, multiple active agents are loaded into the same lipid nanoparticles. In other embodiments, lipid nanoparticles comprising an active agent are used in combination with one or more free drugs. In particular embodiments, LNP compositions comprising an active agent are formed individually and subsequently combined with other compounds for a single co-administration. Alternatively, certain therapies are administered sequentially in a predetermined order. Accordingly, LNP compositions of the instant disclosure may comprise one or more active agents.

Other combination therapies known to those of skill in the art can be used in conjunction with the methods of the instant disclosure.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Preparation of Mixed Lipid Nanoparticle (LNP) Formulations Using a Microfluidic Approach

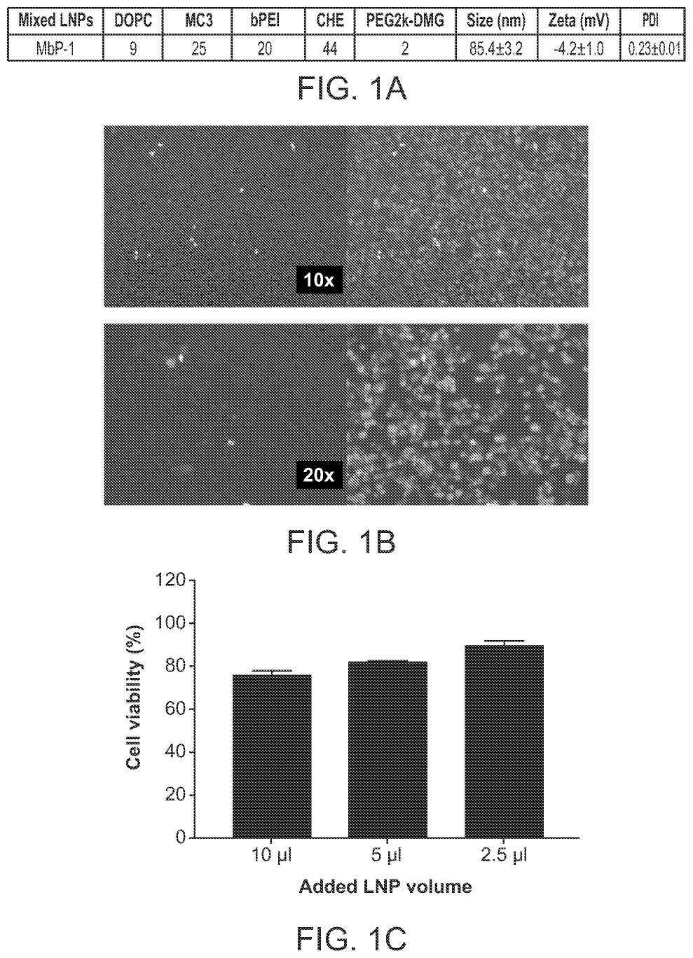

Formulations of LNPs were prepared using ionizable lipids, cationic lipids, non-cationic lipids (e.g., as helper lipids), cholesterol (e.g., as structural lipids) and optionally conjugated lipids (e.g., PEG-lipids) for encapsulating oligonucleotides as cargo. In pilot phase formulations, a validated and widely accepted ionizable lipid, heptatriaconta-6,9,28,31-tetraen-19-yl 4-(dimethylamino)butanoate (MC3), was used as the first component and combined with a cationic branched polyethyleneimine (bPEI) as the second component. The second component, bPEI has a branched structure which has more than one positively charged nitrogen atom per molecule, as compared to one positively charged nitrogen per MC3 molecule. These two components were mixed with other types of lipids across a range of molar ratios (see FIG. 1A) to form an organic phase of formulated LNPs in ethyl alcohol (EtOH). LNPs were also fluorescently labeled by including a Cy5-lipid conjugate in the organic phase. For oligonucleotide cargo, a large circular plasmid with 8870 bp that encodes a nucleic acid modulating controller and a GFP protein, was mixed in pH 4.5 citric acid buffer (50 mM) at 0.27 mg/ml concentration. The mixing of two phases and LNP preparation was performed employing a 2:1 volume ratio (aqueous:organic) using a microfluidic mixing device. LNPs were used without further purification and concentration. Particle size, zeta potential and polydispersity index (PDI) were measured with DLS-PALS and characterization results were tabulated (FIG. 1A). 100 nm particle size mixed LNPs were able to be prepared successfully using the given formulation and lipids. Hepa 1-6 murine hepatocellular carcinoma cells were seeded into 96-well black-walled microplates at 20,000 cells/well/100 μl. Cells were incubated at 37° C. under 5% CO₂ and allowed to incubate and attach overnight. The cells were then treated the following day with a formulation of mixed LNPs, labelled as MbP, using three different volumes of LNPs, corresponding to 500-125 ng plasmid/well, in a total of 100 μl cell culture medium/well. Cells were treated 48 h continuously with the formulations. Then the wells were washed with phenol-red free complete cell culture medium, and cell nuclei were stained with Hoechst. Imaging was performed to detect Cy5 (red), Hoechst (blue) and GFP (green) in live cells. Successful transfection of Hepa 1.6 cells after 48 h treatment was observed (FIG. 1). 100% of treated cells were Cy5 positive (FIG. 1B), which indicated that these cells were LNP positive. Mixed LNPs of the instant disclosure were also able to transfect cells with the large plasmid cargo, unlike the MC3-based LNPs assayed in parallel. Mixed LNPs of the instant Example also did not exert significant cytotoxicity on the cells at tested concentrations, which indicated that the addition of bPEI did not have a negative effect on safety of MbP mixed LNPs (FIG. 1C).

### Example 2: Mixed Cationic Lipid Particles were Identified as Effective for Transfecting Historically Hard-to-Transfect Cell Lines

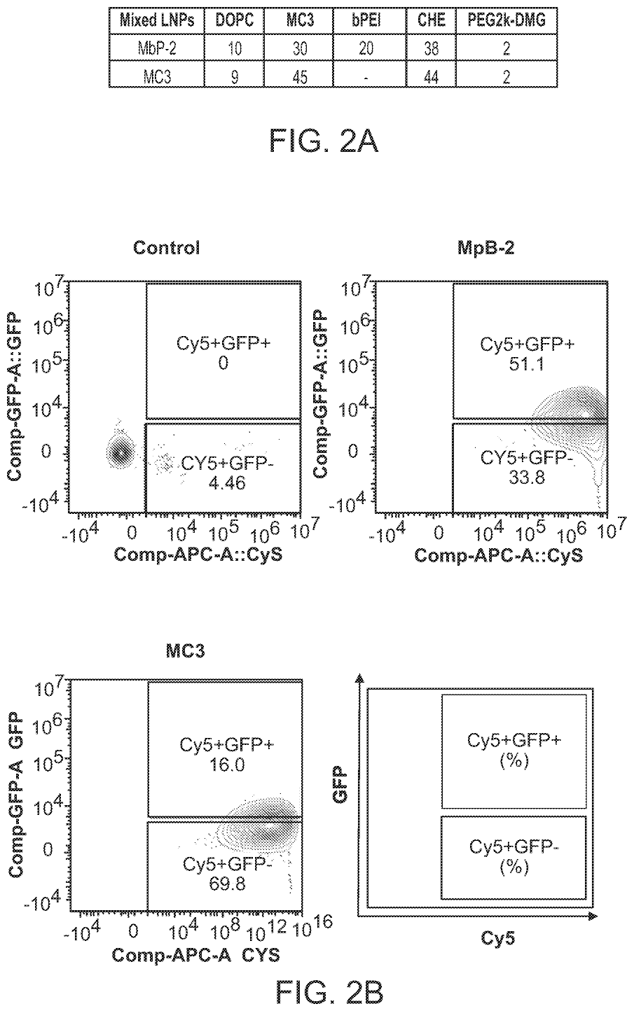

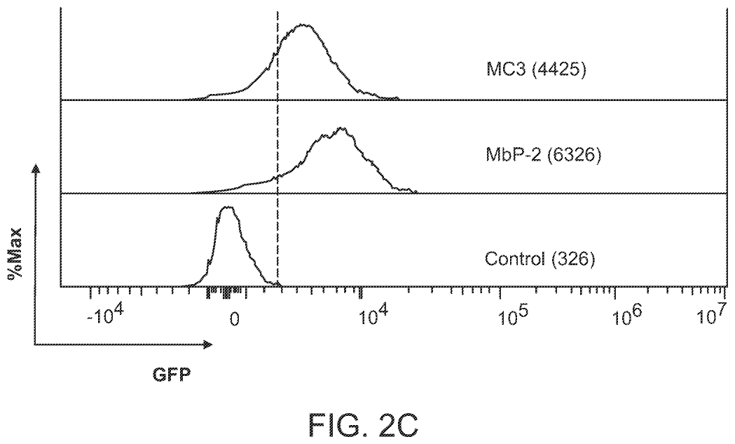

Primary T-cells are historically hard to transfect with non-viral vectors, and they show very low endocytosis of polyplexes. Moreover, intracellular pH of the T cells is higher than other cell types and closer to the pKa of most ionizable lipids, such as MC3 (pKa: 6.4). Without wishing to be bound by theory, this is believed to cause diminished positive charge conversion in the endosomal compartments upon internalization of LNPs, and therefore appears to prevent proton sponge effects and endosomal escape. Use of highly cationic lipids and polymers for transfection can also induce cytotoxicity and autophagosome formation. In an initial attempt to evaluate mixed particles and their performance against the above stated challenges, a formulation of mixed LNPs, labelled as “MbP-2”, consisting of MC3, bPEI, DOPC, cholesterol and PEG2k-DMG was developed (ratios shown in FIG. 2A). A MC3-only formulation at corresponding ratios of other lipid components was also prepared and used as a baseline formulation for comparison. As a representative oligonucleotide cargo, GFP mRNA was encapsulated in both forms of LNP and properties/capabilities of the respective LNP formulations were evaluated. While both MbP-2 and MC3 formulations caused 100% Cy5 positive cells, indicative of both LNPs being successfully internalized, the MbP-2 mixed LNP formulation was observed to transfect >50% of the cells with GFP mRNA (FIG. 2B). In contrast, the MC3 formulation was only observed to transfect 16% of the cells under the same conditions. As a result, the MbP-2 mixed LNP formulation generated 1.5-fold increased GFP signal in the transfected cells as compared to the MC3-alone LNP, as well as 20-fold increased GFP signal in the transfected cells, as compared to control-treated cells (FIG. 2C). These results demonstrated the effectiveness of mixed LNPs for encapsulation and delivery of mRNAs into cells, as compared to MC3-alone LNPs.

### Example 3: Preparation of Mixed C12-200/DOTAP Particles with Different Formulation Parameters

A series of DOTAP/C12-200 mixed lipid nanoparticles (“DC LNPs”) that mixed C12-200 and DOTAP lipids at varying relative concentrations was prepared, thereby introducing both unsaturated and saturated lipid chains into individual LNP formulations. DC LNPs were produced using a microfluidic mixing process. Lipid stocks of DOTAP, C12-200, DOPE, CHE and PEG2k-DMG were prepared in ethanol at concentrations from 20 to 80 mg/mL. The variation in percentage molarity (% mol) of DOPE, DOTAP and PEG were investigated while keeping the percentage molarity (% mol) of C12-200 and total lipid to mRNA mass ratio constant. The concentration of C12-200 was kept below 20% mol to mitigate against predicted toxicity at higher C12-200 levels. mCherry mRNA (996 nucleotides in length) was used as the nucleic acid cargo in an aqueous phase at 0.25 mg/mL concentration. Mixing of the organic and aqueous phases was performed using a 3:1 (aqueous to organic) volume ratio at 8 mL/min flow rate. The characterization parameters of the formulations are summarized in FIG. 3A.

High positive net charge of C12-200 allowed for precise control of particle size, with variation between 72 and 97 nm across 11 out of 12 formulations tested. Surface charge was kept between 4 and 15 mV and PDI below 0.2. PEG2k-DMG was included to increase the stability of the formulations, as well as to provide such particles with predicted stealth properties upon systemic administration.

### Example 4: Mixed C12-200/DOTAP Particles Transfected Lung Cancer Cell Line A549

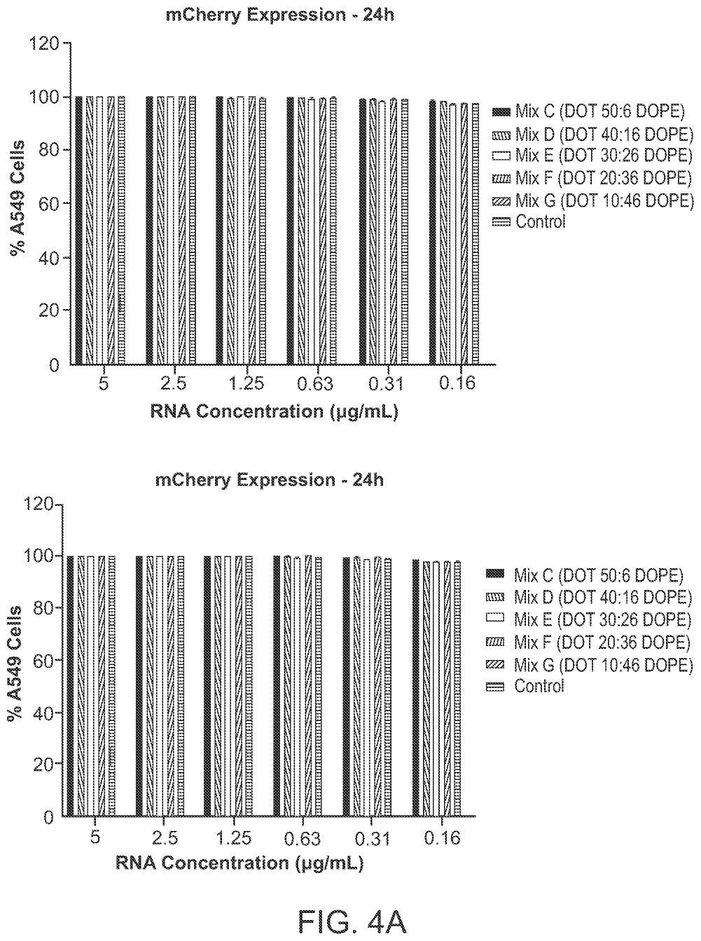

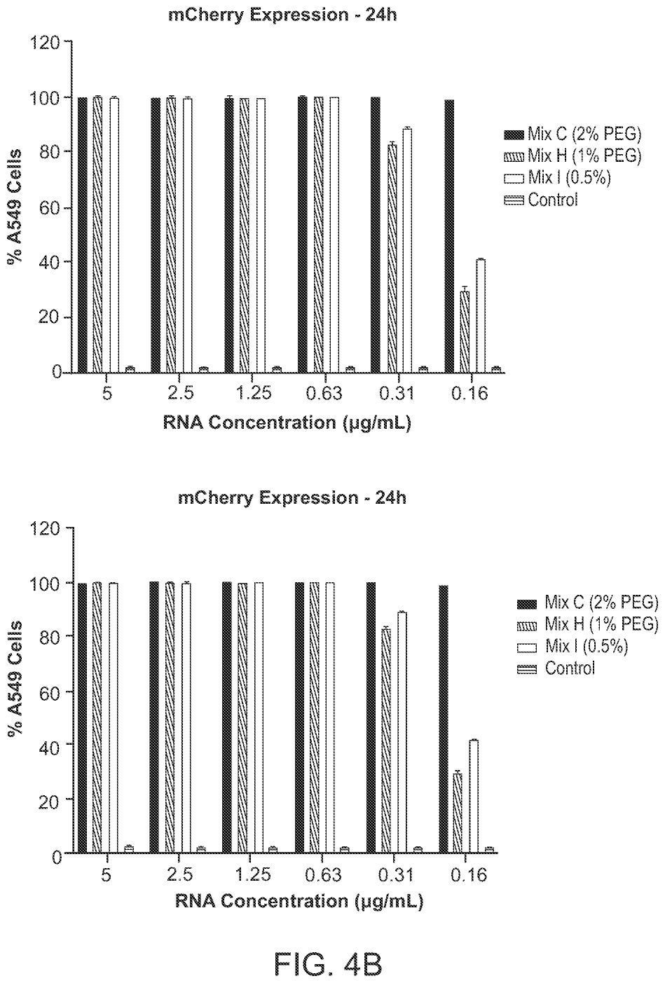

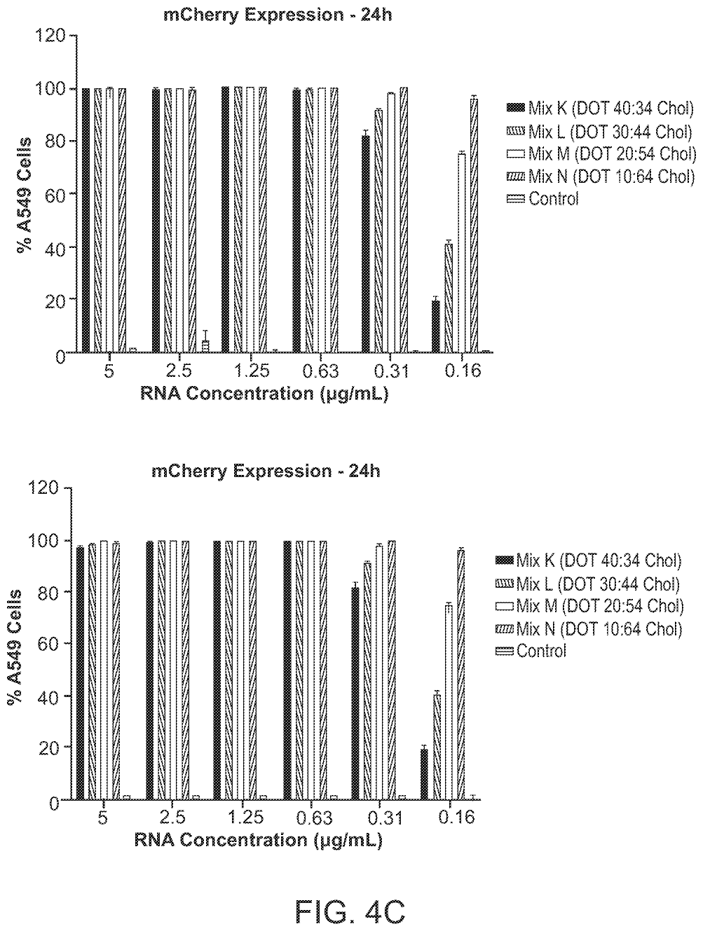

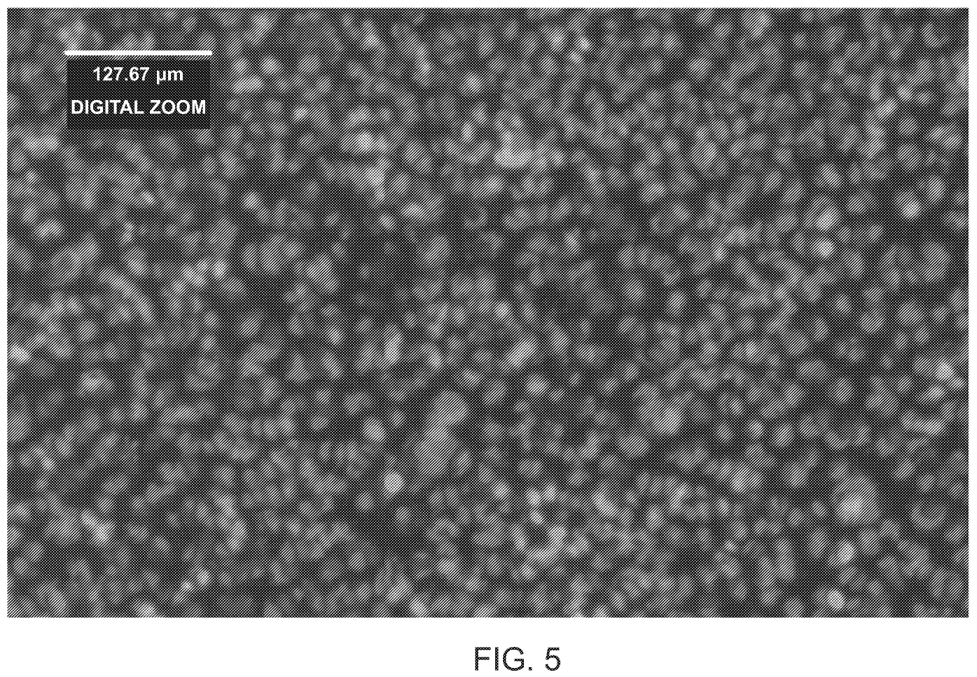

A549 human lung cancer cells were treated in vitro with selected mixed C12-200/DOTAP particles (FIG. 3A). mCherry mRNA transfection was induced in 20,000 cells per well treated with mixed C12-200/DOTAP particles for 24 h at 37° C., 5% CO₂. Dosing of mRNA varied from 5 to 0.16 μg/mL in complete medium. Cytotoxicity of the selected formulations was initially assessed. Cells were specifically treated with mixed C12-200/DOTAP particles under three different experimental conditions: (i) maintaining percentage molarity of C12-200 at 18% while the percentage molarities of DOTAP and DOPE were altered (FIG. 3B), to investigate the impact of the percentage molarity of DOTAP, (ii) maintaining the percentage molarity C12-200 at 18% and varying PEG-lipid ratios (FIG. 3C), and (iii) maintaining the percentage molarity C12-200 at 18% and varying the percentage molarities of DOTAP and cholesterol (FIG. 3D), to investigate the respective roles of DOPE and cholesterol in the specific particles. 24 h treatments revealed that the maximum tolerated mRNA dose in assayed particles was 0.63 μg/ml. Percentages of transfected cells observed for the various particles were also assessed (FIGS. 4A-4C). All mixed C12-200/DOTAP particles transfected 100% of the cells at tolerated dose levels (where at least 80% of cells were viable). Results further indicated that, when the percentage molarity of C12-200 was kept constant and reduced levels of DOTAP % were offset by increased cholesterol concentrations, the transfection efficacy of such lower DOTAP particles dropped at low treatment concentrations. It was also identified that including PEG-lipid in the formulation increased the transfection efficiency, likely by enhancing particle stability. A detailed microscopic analysis of treated cells at the lowest tested concentration was also performed, which revealed that all cells clearly expressed mCherry reporter protein, even at a very low mRNA concentration (FIG. 5).

### Example 5: Mixed C12-200/DOTAP Particles were Stable after Storage at −80° C.

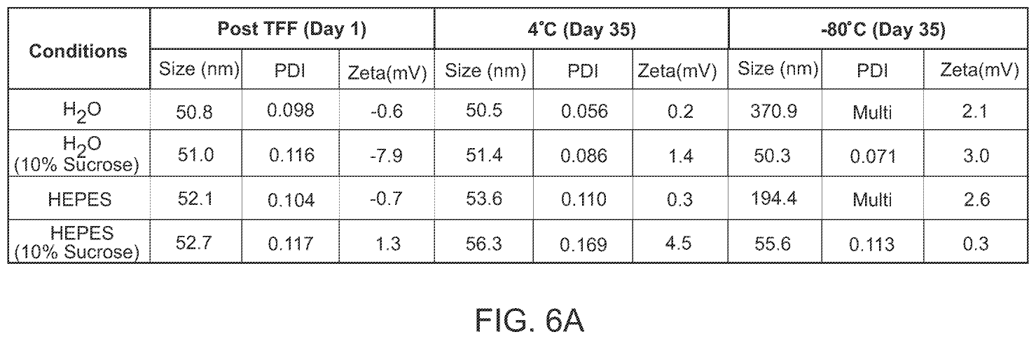

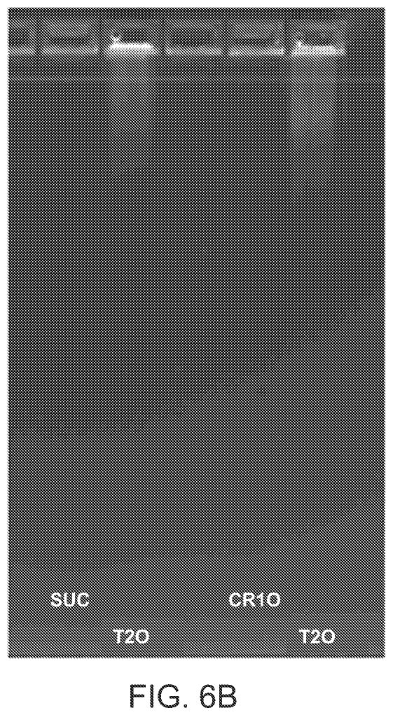

Mixed C12-200/DOTAP particles were produced using a microfluidic mixing process as described above. To prepare the mixed C12-200/DOTAP particles, lipid stocks of DOTAP, C12-200, DOPE, CHE and PEG-DMG were prepared in ethanol at concentrations from 20 to 80 mg/mL. Final lipid concentrations in the particles were respectively maintained at 50, 18, 6, 24 and 2% mol for DOTAP, C12-200, DOPE, cholesterol (CHE) and PEG-DMG. A mRNA of 4598 nucleotides in length that encodes for a nucleic acid modulating controller (i.e. that encodes for protein controller components) was used as the nucleic acid cargo in an aqueous phase at 0.20 mg/mL concentration. Mixing of the organic and aqueous phases was performed using 3:1 (aqueous to organic) volume ratio at 8 mL/min flow rate in a microfluidic flow path design that allowed increased working volumes, as compared to a conventional staggered herringbone model. Resulting particles were subjected to purification and buffer exchange by tangential flow filtration in water or 4 mM HEPES buffer pH 6.5. The particles were stored as is, or with addition to final 10% sucrose, for 35 days at 4° C. and −80° C. Sucrose acts as a cryoprotectant that can preserve the structure of particles during the freezing process. Characterization parameters of the particle formulations before and after storage were determined (FIG. 6A). All formulations remained stable at 4° C. with or without the addition of sucrose. If frozen, only the LNPs with cryoprotectant (here, 10% sucrose) had their characteristics preserved. Frozen storage did not show evidence of premature mRNA release or instability upon thawing (FIG. 6B). The particles of the instant disclosure are therefore likely suitable for cryo-storage and possess a strong internal structure that can provide protection for a cargo nucleic acid (including a long mRNA or nucleic acid modulatory controller cargo).

### Example 6: Mixed C12-200/DOTAP Particles Altered Tropism Relative to DOTAP-Only Particles, Enabling Liver Targeting

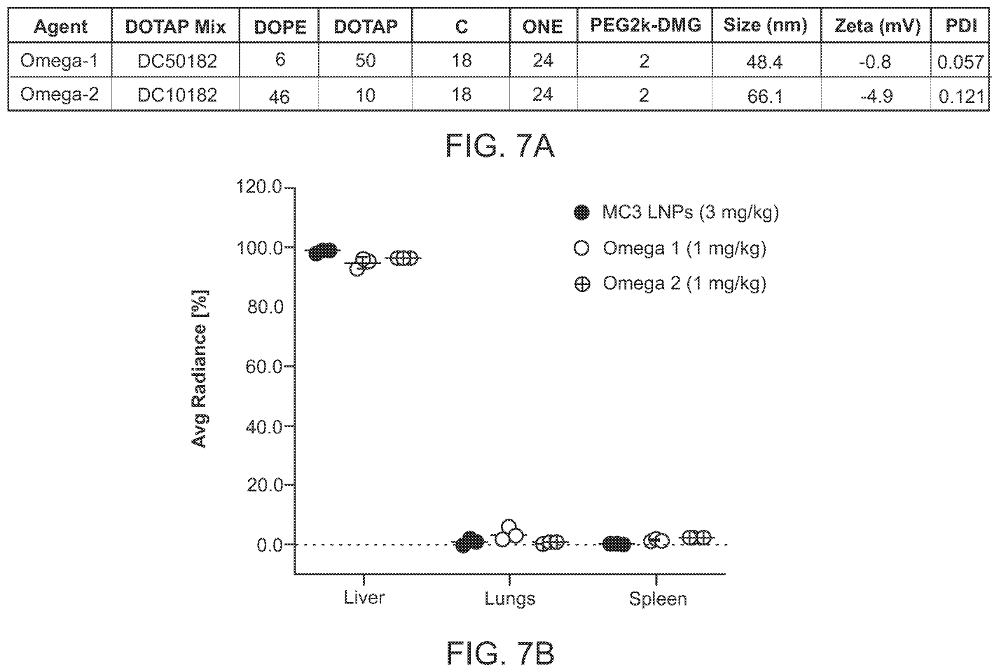

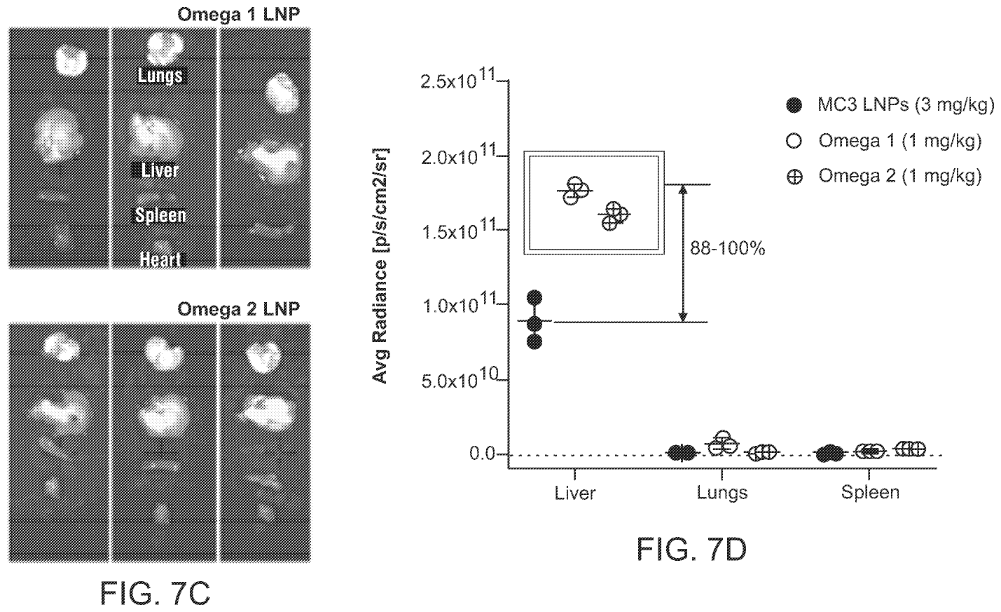

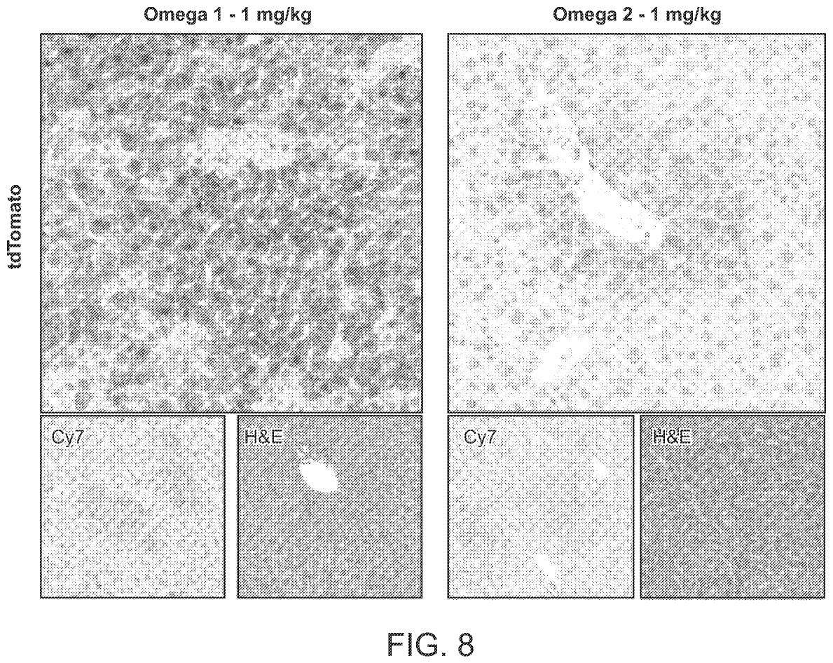

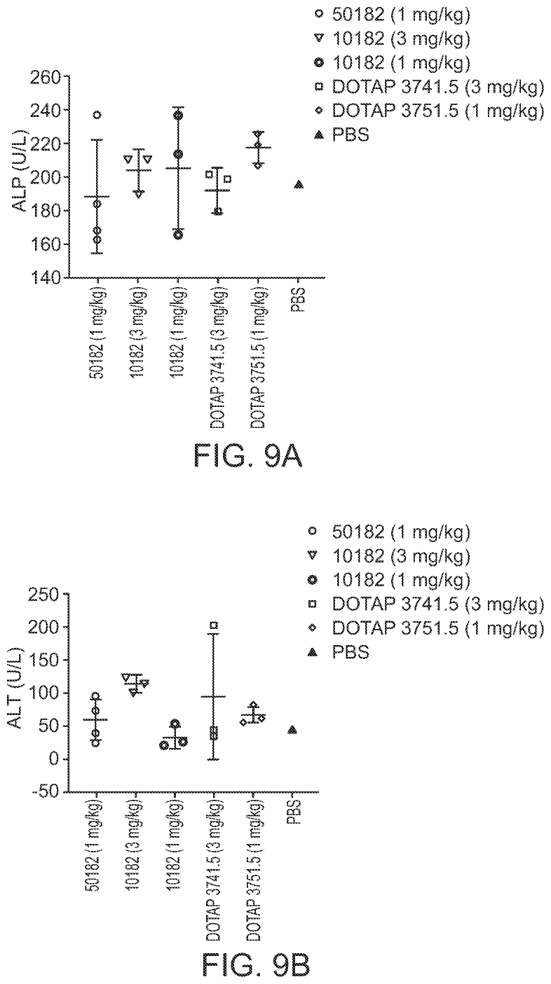

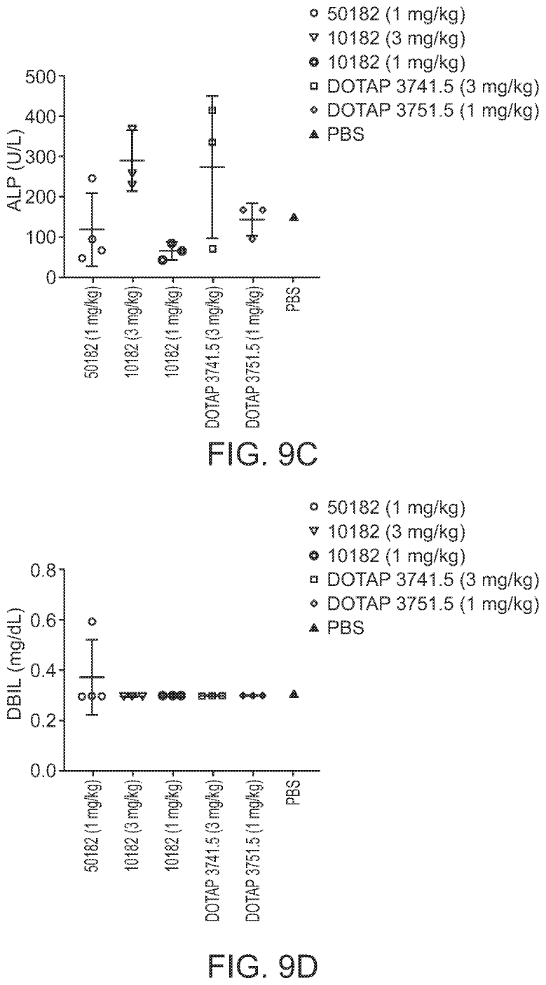

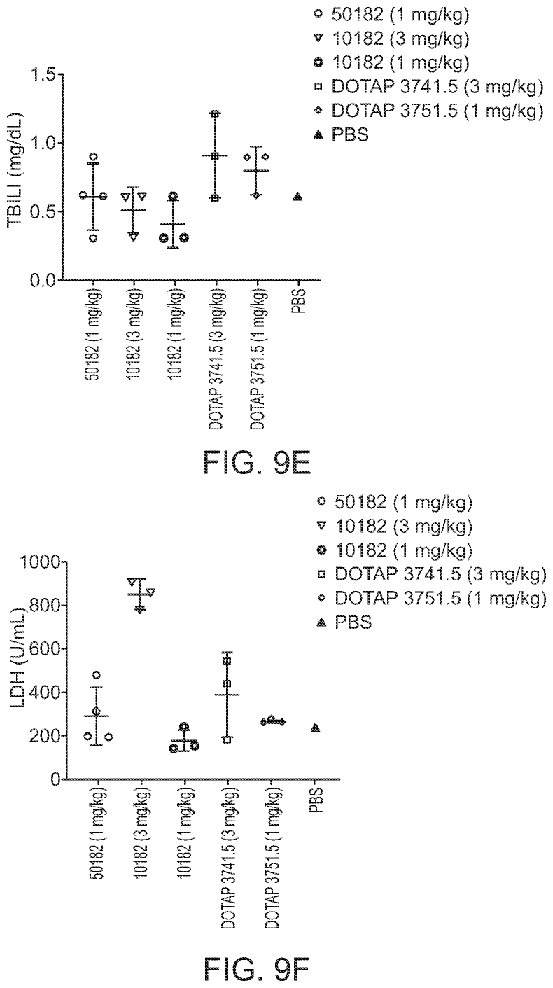

To determine efficacy of the particles of the disclosure for liver tissue targeting, Ail4 mice, which require cargo nucleic acid activity in the nucleus to produce signal, were utilized, and the biodistribution of mixed C12-200/DOTAP particles was evaluated in Ail4 mice (B6.Cg-Gt(ROSA)26Sor^{tm14(CAG-tdTomato)Hze}/J; Ail4 is a Cre reporter tool strain designed to have a loxP-flanked STOP cassette preventing transcription of a CAG promoter-driven red fluorescent protein variant (tdTomato) which is all inserted into the Gt(ROSA)26Sor locus; Ail4 mice express robust tdTomato fluorescence following Cre-mediated recombination (Cre recombinase enzyme (coded by mCre) shows its activity by catalyzing site-specific recombination of DNA between loxP sites)). High and low % mol of DOTAP (50 and 10%) were tested, to investigate its contribution to organ distribution of the particles. It was previously confirmed that DOTAP-only particles with 50% mol DOTAP provided strong and highly organ-restricted activity in the lungs. In the instant biodistribution studies, the impact of the inclusion of saturated lipid chain C12-200 upon organ tropism of the instant particles was evaluated while the DOTAP mol % was maintained at 50%. Particles of the instant disclosure were fluorescently labeled with Cy7-DOPE (0.25% mol) and loaded with mCre mRNA. The particle formulations were administered at 1 mg/kg dose via an intravenous route. The instant particles were compared to a distinct liver-targeting formulation (an MC3-containing formulation loaded with mCre), which was also prepared and administered at 3 mg/kg dose. 48 h post-administration, ex vivo organ imaging for tdTomato was performed. Highly specific liver activity was observed for all formulations (FIGS. 7A-7C). The mixed C12-200/DOTAP particles of the instant disclosure were able to convert the specific activity of DOTAP-only LNPs from lungs to liver. The extent of protein expression observed for mixed C12-200/DOTAP particles was 80 to 100% above the effect observed for MC3-containing particles, which were used as control in a 3× higher dose (FIG. 7D). Immunohistochemistry images of liver samples confirmed tdTomato production and Cy7-labeled particle accumulation (both stained in brown; FIG. 8). Further, liver function tests confirmed the safety of assayed mixed lipid particles at 1 mg/kg mRNA dose (FIGS. 9A-9F).

### Example 7: Mixed C12-200/DOTAP Particles Effectively Deliver Nucleic Acid Modulating Controllers into Liver Tissue

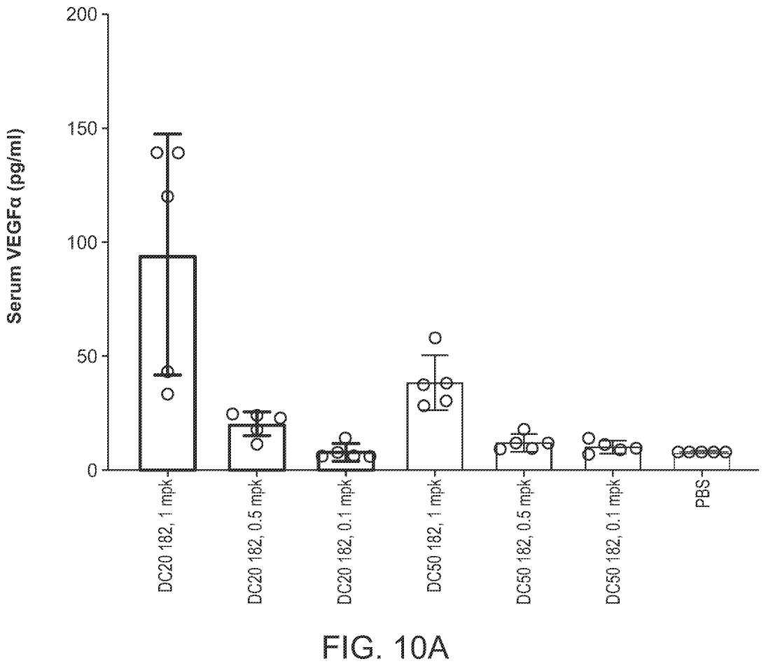

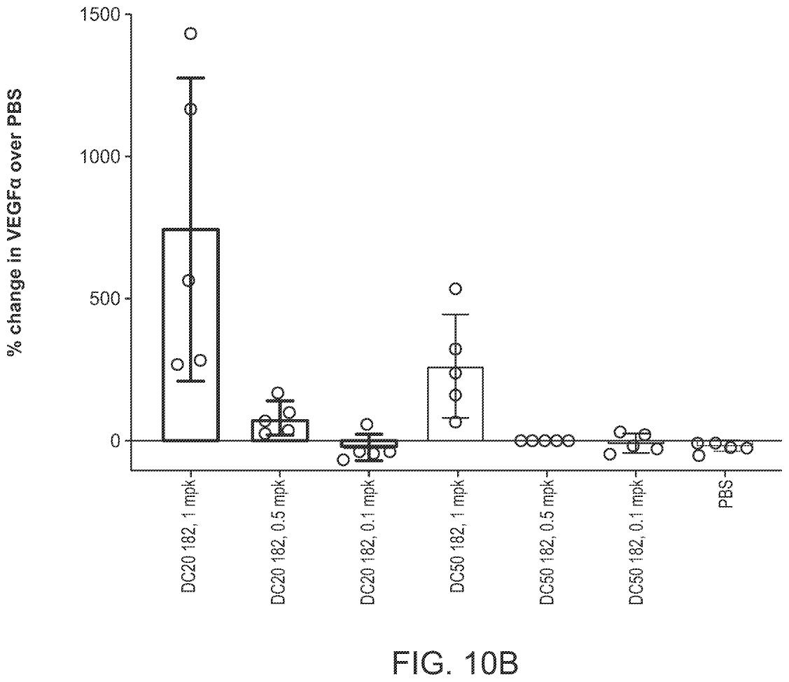

Having evaluated the biodistribution of mixed C12-200/DOTAP particles, such particles harboring a nucleic acid modulating controller were evaluated for in vivo efficacy in liver tissue. In particular, wild type C57B16/J mice were treated with two different mixed C12-200/DOTAP particle formulations, each harboring a VEGFa mRNA. Intravenous administration to mice of these formulations at three distinct dose levels showed that both tested forms of mixed C12-200/DOTAP particle increased serum VEGFa levels significantly, especially at a 1 mg/kg dose level, as compared to an appropriate control (FIGS. 10A-10B). These data further confirmed the efficacy of the mixed C12-200/DOTAP particles of the instant disclosure in encapsulating large nucleic acids, including nucleic acid-modulating controllers, and delivering them to the liver with high selectivity, specificity and efficacy, thereby generating a physiological response at even low dose levels.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A nucleic acid-lipid particle for delivering a nucleic acid cargo to a liver tissue of a subject, the nucleic acid-lipid particle comprising:
1,2-dioleoyl-3-trimethylammonium-propane (DOTAP) comprising from about 10 mol % to about 50 mol % of the total lipid present in the nucleic acid-lipid particle; and
ionizable lipid comprising from about 5 mol % to about 50 mol % of the total lipid present in the nucleic acid-lipid particle.

2. The nucleic acid-lipid particle of claim 1, wherein the ionizable lipid is selected from the group consisting of 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), N4-Cholesteryl-Spermine HCl (GL67), N1-[2-((1S)-1-[(3-aminopropyl)amino]-4-[di(3-amino-propyl)amino]butylcarboxamido)ethyl]-3,4-di[oleyloxy]-benzamide (MVL5), and polymeric branched polyethyleneimine (bPEI).

3. The nucleic acid-lipid particle of claim 1 comprising one or more non-cationic lipids comprising from about 25 mol % to about 85 mol % of the total lipid present in the lipid-nucleic acid particle, optionally wherein the one or more non-cationic lipids comprise a structural lipid selected from the group consisting of cholesterol, β-sitosterol and derivatives thereof.

4. The nucleic acid-lipid particle of claim 1 comprising cholesterol, β-sitosterol or derivatives thereof from about 10 mol % to about 75 mol % of the total lipid present in the nucleic acid-lipid particle, optionally comprising cholesterol, β-sitosterol or derivatives thereof from about 20 mol % to about 65 mol % of the total lipid present in the nucleic acid-lipid particle, optionally comprising cholesterol or a derivative thereof from about 24 mol % to about 64 mol % of the total lipid present in the nucleic acid-lipid particle, optionally comprising cholesterol, β-sitosterol or derivatives thereof at a level selected from the group consisting of about 24 mol % of the total lipid present in the nucleic acid-lipid particle, about 34 mol % of the total lipid present in the nucleic acid-lipid particle, about 38 mol % of the total lipid present in the nucleic acid-lipid particle, about 44 mol % of the total lipid present in the nucleic acid-lipid particle, about 54 mol % of the total lipid present in the nucleic acid-lipid particle and about 64 mol % of the total lipid present in the nucleic acid-lipid particle.

5. The nucleic acid-lipid particle of claim 1 comprising one or more non-cationic lipid other than cholesterol, β-sitosterol or a derivative thereof, optionally wherein the one or more non-cationic lipid other than cholesterol, β-sitosterol or derivatives thereof comprises from about 5 mol % to about 50 mol % of the total lipid present in the lipid-nucleic acid particle, optionally wherein the one or more non-cationic lipid other than cholesterol, β-sitosterol or derivatives thereof comprises from about 5 mol % to about 30 mol % of the total lipid present in the lipid-nucleic acid particle, optionally wherein the one or more non-cationic lipid other than cholesterol, β-sitosterol or derivatives thereof comprises from about 5 mol % to about 10 mol % of the total lipid present in the lipid-nucleic acid particle.

6. The nucleic acid-lipid particle of claim 1 comprising one or more non-cationic lipid other than cholesterol, β-sitosterol or derivatives thereof at a level selected from the group consisting of about 6 mol % of the total lipid present in the nucleic acid-lipid particle, about 7 mol % of the total lipid present in the nucleic acid-lipid particle, about 7.5 mol % of the total lipid present in the nucleic acid-lipid particle, about 8 mol % of the total lipid present in the nucleic acid-lipid particle, about 9 mol % of the total lipid present in the nucleic acid-lipid particle, about 10 mol % of the total lipid present in the nucleic acid-lipid particle, about 16 mol % of the total lipid present in the nucleic acid-lipid particle, about 26 mol % of the total lipid present in the nucleic acid-lipid particle, about 36 mol % of the total lipid present in the nucleic acid-lipid particle and about 46 mol % of the total lipid present in the nucleic acid-lipid particle.

7. The nucleic acid-lipid particle of claim 5, wherein the one or more non-cationic lipid other than cholesterol, β-sitosterol or derivatives thereof comprises a non-cationic lipid selected from the group consisting of 1,2-Dioleoyl-sn-glycero-3-phosphoethanolamine (DOPE), 1,2-dioleoyl-sn-glycero-3-phosphocholine (DOPC), and 1,2-Distearoyl-sn-glycero-3-phosphocholine (DSPC), optionally wherein the one or more non-cationic lipid other than cholesterol, β-sitosterol or derivatives thereof is 1,2-Dioleoyl-sn-glycero-3-phosphoethanolamine (DOPE).

8. The nucleic acid-lipid particle of claim 1, wherein the nucleic acid-lipid particle does not comprise a PEG-lipid conjugate, optionally wherein the nucleic acid-lipid particle does not comprise PEG.

9. The nucleic acid-lipid particle of claim 8, wherein the nucleic acid-lipid particle is a component of a multi-dose therapy.

10. The nucleic acid-lipid particle of claim 1 comprising a conjugated lipid that inhibits aggregation of particles comprising from 0.01 to 3% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at a level selected from the group consisting of about 0.5 mol % of the total lipid present in the nucleic acid-lipid particle, about 1.0 mol % of the total lipid present in the nucleic acid-lipid particle, about 1.5 mol % of the total lipid present in the nucleic acid-lipid particle, and about 2.0 mol % of the total lipid present in the nucleic acid-lipid particle.

11. The nucleic acid-lipid particle of claim 1, wherein the nucleic acid cargo comprises a synthetic or naturally occurring RNA or DNA, or derivatives thereof, optionally wherein the nucleic acid cargo is a modified RNA, optionally wherein the modified RNA is selected from the group consisting of a modified mRNA, a modified antisense oligonucleotide and a modified siRNA, optionally wherein the modified mRNA encodes a nucleic acid modulating controller.

12. The nucleic acid-lipid particle of claim 1, wherein:
the nucleic acid cargo comprises one or more modifications selected from the group consisting of 2′-O-methyl modified nucleotides, a nucleotide comprising a 5′-phosphorothioate group, a terminal nucleotide linked to a cholesteryl derivative, a 2′-deoxy-2′-fluoro modified nucleotide, a 5′-methoxy-modified nucleotide (e.g., 5′-methoxyuridine), a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide; internucleoside linkages or backbones including phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; and/or
the liver tissue is selected from the group consisting of hepatocytes, vascular cells (i.e. hepatic sinusoids), hepatic stellate cells, endothelial cells, fibroblasts, mesenchymal cells, immune cells, cancer cells, Kupffer cells, astrocytes, oval-shaped vascular endothelial cells, liver-derived stem/progenitor cells, and stem/progenitor or cancer cells derived from non-liver tissue.

13. (canceled)

14. The nucleic acid-lipid particle of claim 1 comprising DOTAP at a level selected from the group consisting of about 10 mol % of the total lipid present in the nucleic acid-lipid particle, about 20 mol % of the total lipid present in the nucleic acid-lipid particle, about 30 mol % of the total lipid present in the nucleic acid-lipid particle, about 40 mol % of the total lipid present in the nucleic acid-lipid particle and about 50 mol % of the total lipid present in the nucleic acid-lipid particle.

15. A nucleic acid-lipid particle for delivering a nucleic acid cargo to a liver tissue of a subject, selected from among:
A nucleic acid-lipid particle comprising:
1,2-dioleoyl-3-trimethylammonium-propane (DOTAP) comprising about 50 mol % of the total lipid present in the nucleic acid-lipid particle; and
ionizable lipid comprising about 18 mol % of the total lipid present in the nucleic acid-lipid particle;
A nucleic acid-lipid particle comprising:
1,2-dioleoyl-3-trimethylammonium-propane (DOTAP) comprising about 40 mol % of the total lipid present in the nucleic acid-lipid particle; and
ionizable lipid comprising about 18 mol % of the total lipid present in the nucleic acid-lipid particle;
A nucleic acid-lipid particle comprising:
1,2-dioleoyl-3-trimethylammonium-propane (DOTAP) comprising about 30 mol % of the total lipid present in the nucleic acid-lipid particle; and
ionizable lipid comprising about 18 mol % of the total lipid present in the nucleic acid-lipid particle;
A nucleic acid-lipid particle comprising:
1,2-dioleoyl-3-trimethylammonium-propane (DOTAP) comprising about 20 mol % of the total lipid present in the nucleic acid-lipid particle; and
ionizable lipid comprising about 18 mol % of the total lipid present in the nucleic acid-lipid particle;
A nucleic acid-lipid particle comprising:
1,2-dioleoyl-3-trimethylammonium-propane (DOTAP) comprising about 10 mol % of the total lipid present in the nucleic acid-lipid particle; and
ionizable lipid comprising about 18 mol % of the total lipid present in the nucleic acid-lipid particle;
A nucleic acid-lipid particle comprising:
1,2-dioleoyl-3-trimethylammonium-propane (DOTAP) comprising about 50 mol % of the total lipid present in the nucleic acid-lipid particle;
ionizable lipid comprising about 18 mol % of the total lipid present in the nucleic acid-lipid particle; and
one or more non-cationic lipid other than cholesterol, β-sitosterol or derivatives thereof present at about 7.0 mol % of the total lipid present in the nucleic acid-lipid particle;
A nucleic acid-lipid particle comprising:
1,2-dioleoyl-3-trimethylammonium-propane (DOTAP) comprising about 50 mol % of the total lipid present in the nucleic acid-lipid particle;
ionizable lipid comprising about 18 mol % of the total lipid present in the nucleic acid-lipid particle; and
one or more non-cationic lipid other than cholesterol, β-sitosterol or derivatives thereof at about 7.5 mol % of the total lipid present in the nucleic acid-lipid particle;
A nucleic acid-lipid particle comprising:
1,2-dioleoyl-3-trimethylammonium-propane (DOTAP) comprising about 50 mol % of the total lipid present in the nucleic acid-lipid particle;
ionizable lipid comprising about 18 mol % of the total lipid present in the nucleic acid-lipid particle; and
one or more non-cationic lipid other than cholesterol, β-sitosterol or derivatives thereof at about 8.0 mol % of the total lipid present in the nucleic acid-lipid particle;
A nucleic acid-lipid particle comprising:
1,2-dioleoyl-3-trimethylammonium-propane (DOTAP) comprising about 40 mol % of the total lipid present in the nucleic acid-lipid particle;
ionizable lipid comprising about 18 mol % of the total lipid present in the nucleic acid-lipid particle; and
cholesterol, β-sitosterol or derivatives thereof at about 34 mol % of the total lipid present in the nucleic acid-lipid particle;
A nucleic acid-lipid particle comprising:
1,2-dioleoyl-3-trimethylammonium-propane (DOTAP) comprising about 30 mol % of the total lipid present in the nucleic acid-lipid particle;
ionizable lipid comprising about 18 mol % of the total lipid present in the nucleic acid-lipid particle; and
cholesterol, β-sitosterol or derivatives thereof at about 44 mol % of the total lipid present in the nucleic acid-lipid particle;
A nucleic acid-lipid particle comprising:
1,2-dioleoyl-3-trimethylammonium-propane (DOTAP) comprising about 20 mol % of the total lipid present in the nucleic acid-lipid particle;
ionizable lipid comprising about 18 mol % of the total lipid present in the nucleic acid-lipid particle; and
cholesterol, β-sitosterol or derivatives thereof at about 54 mol % of the total lipid present in the nucleic acid-lipid particle; and/or
A nucleic acid-lipid particle comprising:
1,2-dioleoyl-3-trimethylammonium-propane (DOTAP) comprising about 10 mol % of the total lipid present in the nucleic acid-lipid particle;
ionizable lipid comprising about 18 mol % of the total lipid present in the nucleic acid-lipid particle; and
cholesterol, β-sitosterol or derivatives thereof at about 64 mol % of the total lipid present in the nucleic acid-lipid particle.

16. The nucleic acid-lipid particle of claim 15:
wherein the ionizable lipid is 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200);
comprising cholesterol, β-sitosterol or derivatives thereof at about 24 mol % of the total lipid present in the nucleic acid-lipid particle, optionally further comprising one or more non-cationic lipid other than cholesterol, β-sitosterol or derivatives thereof at about 6 mol % of the total lipid present in the nucleic acid-lipid particle, optionally wherein the one or more non-cationic lipid other than cholesterol, β-sitosterol or derivatives thereof is 1,2-Dioleoyl-sn-glycero-3-phosphoethanolamine (DOPE), optionally further comprising a polyethyleneglycol (PEG)-lipid conjugate, optionally wherein the PEG-lipid conjugate comprises about 2.0 mol % of the total lipid present in the nucleic acid-lipid particle, optionally wherein the PEG-lipid conjugate is 1,2-dimyristoyl-rac-glycero-3-methoxypolyethylene glycol-2000 (DMG-PEG2k);
comprising cholesterol, β-sitosterol or derivatives thereof at about 24 mol % of the total lipid present in the nucleic acid-lipid particle, optionally further comprising one or more non-cationic lipid other than cholesterol, β-sitosterol or derivatives thereof at about 16 mol % of the total lipid present in the nucleic acid-lipid particle, optionally wherein the one or more non-cationic lipid other than cholesterol, β-sitosterol or derivatives thereof is 1,2-Dioleoyl-sn-glycero-3-phosphoethanolamine (DOPE), optionally further comprising a polyethyleneglycol (PEG)-lipid conjugate, optionally wherein the PEG-lipid conjugate comprises about 2.0 mol % of the total lipid present in the nucleic acid-lipid particle, optionally wherein the PEG-lipid conjugate is 1,2-dimyristoyl-rac-glycero-3-methoxypolyethylene glycol-2000 (DMG-PEG2k);
comprising cholesterol, β-sitosterol or derivatives thereof at about 24 mol % of the total lipid present in the nucleic acid-lipid particle, optionally further comprising one or more non-cationic lipid other than cholesterol, β-sitosterol or derivatives thereof at about 26 mol % of the total lipid present in the nucleic acid-lipid particle, optionally wherein the one or more non-cationic lipid other than cholesterol, β-sitosterol or derivatives thereof is 1,2-Dioleoyl-sn-glycero-3-phosphoethanolamine (DOPE), optionally further comprising a polyethyleneglycol (PEG)-lipid conjugate, optionally wherein the PEG-lipid conjugate comprises about 2.0 mol % of the total lipid present in the nucleic acid-lipid particle, optionally wherein the PEG-lipid conjugate is 1,2-dimyristoyl-rac-glycero-3-methoxypolyethylene glycol-2000 (DMG-PEG2k);
comprising cholesterol, β-sitosterol or derivatives thereof at about 24 mol % of the total lipid present in the nucleic acid-lipid particle, optionally further comprising one or more non-cationic lipid other than cholesterol, β-sitosterol or derivatives thereof at about 36 mol % of the total lipid present in the nucleic acid-lipid particle, optionally wherein the one or more non-cationic lipid other than cholesterol, β-sitosterol or derivatives thereof is 1,2-Dioleoyl-sn-glycero-3-phosphoethanolamine (DOPE), optionally further comprising a polyethyleneglycol (PEG)-lipid conjugate, optionally wherein the PEG-lipid conjugate comprises about 2.0 mol % of the total lipid present in the nucleic acid-lipid particle, optionally wherein the PEG-lipid conjugate is 1,2-dimyristoyl-rac-glycero-3-methoxypolyethylene glycol-2000 (DMG-PEG2k);
comprising cholesterol, β-sitosterol or derivatives thereof at about 24 mol % of the total lipid present in the nucleic acid-lipid particle, optionally further comprising one or more non-cationic lipid other than cholesterol, β-sitosterol or derivatives thereof at about 46 mol % of the total lipid present in the nucleic acid-lipid particle, optionally wherein the one or more non-cationic lipid other than cholesterol, β-sitosterol or derivatives thereof is 1,2-Dioleoyl-sn-glycero-3-phosphoethanolamine (DOPE), optionally further comprising a polyethyleneglycol (PEG)-lipid conjugate, optionally wherein the PEG-lipid conjugate comprises about 2.0 mol % of the total lipid present in the nucleic acid-lipid particle, optionally wherein the PEG-lipid conjugate is 1,2-dimyristoyl-rac-glycero-3-methoxypolyethylene glycol-2000 (DMG-PEG2k);
comprising cholesterol, β-sitosterol or derivatives thereof at about 24 mol % of the total lipid present in the nucleic acid-lipid particle, optionally wherein the one or more non-cationic lipid other than cholesterol, β-sitosterol or derivatives thereof is 1,2-Dioleoyl-sn-glycero-3-phosphoethanolamine (DOPE), optionally further comprising a polyethyleneglycol (PEG)-lipid conjugate, optionally wherein the PEG-lipid conjugate comprises about 1.0 mol % of the total lipid present in the nucleic acid-lipid particle, optionally wherein the PEG-lipid conjugate is 1,2-dimyristoyl-rac-glycero-3-methoxypolyethylene glycol-2000 (DMG-PEG2k);
further comprising cholesterol, β-sitosterol or derivatives thereof at about 24 mol % of the total lipid present in the nucleic acid-lipid particle, optionally wherein the one or more non-cationic lipid other than cholesterol, β-sitosterol or derivatives thereof is 1,2-Dioleoyl-sn-glycero-3-phosphoethanolamine (DOPE), optionally further comprising a polyethyleneglycol (PEG)-lipid conjugate, optionally wherein the PEG-lipid conjugate comprises about 0.5 mol % of the total lipid present in the nucleic acid-lipid particle, optionally wherein the PEG-lipid conjugate is 1,2-dimyristoyl-rac-glycero-3-methoxypolyethylene glycol-2000 (DMG-PEG2k);
further comprising cholesterol, β-sitosterol or derivatives thereof at about 24 mol % of the total lipid present in the nucleic acid-lipid particle, optionally wherein the one or more non-cationic lipid other than cholesterol, β-sitosterol or derivatives thereof is 1,2-Dioleoyl-sn-glycero-3-phosphoethanolamine (DOPE), optionally wherein the nucleic acid-lipid particle does not comprise a PEG-lipid conjugate, optionally wherein the nucleic acid-lipid particle does not comprise PEG;
further comprising one or more non-cationic lipid other than cholesterol, β-sitosterol or derivatives thereof at about 6.0 mol % of the total lipid present in the nucleic acid-lipid particle, optionally wherein the one or more non-cationic lipid other than cholesterol, β-sitosterol or derivatives thereof is 1,2-Dioleoyl-sn-glycero-3-phosphoethanolamine (DOPE), optionally further comprising a polyethyleneglycol (PEG)-lipid conjugate, optionally wherein the PEG-lipid conjugate comprises about 2.0 mol % of the total lipid present in the nucleic acid-lipid particle, optionally wherein the PEG-lipid conjugate is 1,2-dimyristoyl-rac-glycero-3-methoxypolyethylene glycol-2000 (DMG-PEG2k);
further comprising one or more non-cationic lipid other than cholesterol, β-sitosterol or derivatives thereof at about 6.0 mol % of the total lipid present in the nucleic acid-lipid particle, optionally wherein the one or more non-cationic lipid other than cholesterol, β-sitosterol or derivatives thereof is 1,2-Dioleoyl-sn-glycero-3-phosphoethanolamine (DOPE), optionally further comprising a polyethyleneglycol (PEG)-lipid conjugate, optionally wherein the PEG-lipid conjugate comprises about 2.0 mol % of the total lipid present in the nucleic acid-lipid particle, optionally wherein the PEG-lipid conjugate is 1,2-dimyristoyl-rac-glycero-3-methoxypolyethylene glycol-2000 (DMG-PEG2k);
further comprising one or more non-cationic lipid other than cholesterol, β-sitosterol or derivatives thereof at about 6.0 mol % of the total lipid present in the nucleic acid-lipid particle, optionally wherein the one or more non-cationic lipid other than cholesterol, β-sitosterol or derivatives thereof is 1,2-Dioleoyl-sn-glycero-3-phosphoethanolamine (DOPE), optionally further comprising a polyethyleneglycol (PEG)-lipid conjugate, optionally wherein the PEG-lipid conjugate comprises about 2.0 mol % of the total lipid present in the nucleic acid-lipid particle, optionally wherein the PEG-lipid conjugate is 1,2-dimyristoyl-rac-glycero-3-methoxypolyethylene glycol-2000 (DMG-PEG2k);
further comprising one or more non-cationic lipid other than cholesterol, β-sitosterol or derivatives thereof at about 6.0 mol % of the total lipid present in the nucleic acid-lipid particle, optionally wherein the one or more non-cationic lipid other than cholesterol, β-sitosterol or derivatives thereof is 1,2-Dioleoyl-sn-glycero-3-phosphoethanolamine (DOPE), optionally further comprising a polyethyleneglycol (PEG)-lipid conjugate, optionally wherein the PEG-lipid conjugate comprises about 2.0 mol % of the total lipid present in the nucleic acid-lipid particle, optionally wherein the PEG-lipid conjugate is 1,2-dimyristoyl-rac-glycero-3-methoxypolyethylene glycol-2000 (DMG-PEG2k); and/or
wherein the nucleic acid-lipid particle is administered to treat a liver disease or disorder, optionally wherein the disease or disorder is selected from the group consisting of biliary atresia, Alagille Syndrome, alpha-1 antitrypsin deficiency, Tyrosinemia, neonatal hepatitis, hepatitis C virus infection, hepatitis B virus infection, hepatitis A virus infection, hepatocellular carcinoma and Wilson's disease.

17-50. (canceled)

51. A pharmaceutical composition comprising the nucleic acid-lipid particle of claim 1 and a pharmaceutically acceptable carrier.

52. The pharmaceutical composition of claim 51, wherein:
the pharmaceutical composition is formulated for parenteral administration, optionally for intravenous injection; and/or;
the pharmaceutical composition is formulated for direct injection into the liver tissue.

53-54. (canceled)

55. An injectate comprising the nucleic acid-lipid particle claim 1.

56. A method selected from among:
A method for delivering a nucleic acid cargo to a liver tissue of a subject comprising administering a nucleic acid-lipid particle, pharmaceutical composition, or injectate comprising a nucleic acid-lipid particle comprising 1,2-dioleoyl-3-trimethylammonium-propane (DOTAP) comprising from about 10 mol % to about 50 mol % of the total lipid present in the nucleic acid-lipid particle; and ionizable lipid comprising from about 5 mol % to about 50 mol % of the total lipid present in the nucleic acid-lipid particle to the subject; and/or
A method for treating or preventing a disease or disorder in a subject, the method comprising administering a nucleic acid-lipid particle, pharmaceutical composition, or injectate comprising a nucleic acid-lipid particle comprising 1,2-dioleoyl-3-trimethylammonium-propane (DOTAP) comprising from about 10 mol % to about 50 mol % of the total lipid present in the nucleic acid-lipid particle; and ionizable lipid comprising from about 5 mol % to about 50 mol % of the total lipid present in the nucleic acid-lipid particle to the subject.

57. (canceled)

58. The method of claim 56, wherein:
the nucleic acid-lipid particle, pharmaceutical composition, or injectate is administered intravenously and expression of the nucleic acid cargo in cells of the liver tissue of the subject occurs at a level that is at least two-fold higher than expression of the nucleic acid cargo in cells of lung, heart, and spleen of the subject, optionally wherein expression of the nucleic acid cargo in cells of the liver tissue of the subject is at least three-fold higher, optionally at least four-fold higher, optionally at least five-fold higher, optionally at least six-fold higher, optionally at least seven-fold higher, optionally at least eight-fold higher, optionally at least nine-fold higher, optionally at least ten-fold higher, optionally at least eleven-fold higher, optionally at least twelve-fold higher, optionally at least thirteen-fold higher, optionally at least fourteen-fold higher, optionally at least fifteen-fold higher, optionally at least twenty-fold higher, than expression of the nucleic acid cargo in cells of lung, heart, and spleen of the subject;
the nucleic acid-lipid particle, pharmaceutical composition, or injectate is administered intravenously and the nucleic acid-lipid particle localizes to the liver tissue of the subject at an at least two-fold higher concentration than the concentration of the nucleic acid-lipid particle in one or more other tissues of the subject selected from the group consisting of lung, heart, spleen, ovaries and pancreas, optionally wherein at least three-fold, optionally at least four-fold, optionally at least five-fold, optionally at least six-fold higher concentration of the nucleic acid-lipid particle is present in liver as compared to one or more other tissues of the subject selected from the group consisting of lung, heart, spleen, ovaries and pancreas;
the disease or disorder is selected from the group consisting of: a liver disease or disorder, optionally wherein the liver disease or disorder is selected from the group consisting of biliary atresia, Alagille Syndrome, alpha-1 antitrypsin deficiency, Tyrosinemia, neonatal hepatitis, hepatitis C virus infection, hepatitis B virus infection, hepatitis A virus infection, hepatocellular carcinoma, and Wilson's disease; a joint disease or disorder, optionally wherein the joint disease or disorder is selected from the group consisting of rheumatoid arthritis, psoriatic arthritis, gout, tendinitis, bursitis, Carpal Tunnel Syndrome and osteoarthritis; an inflammatory disease or disorder, optionally wherein the inflammatory disease or disorder is selected from the group consisting of inflammatory bowel disease, peritonitis, osteomyelitis, cachexia, pancreatitis, trauma induced shock, bronchial asthma, allergic rhinitis, cystic fibrosis, acute bronchitis, acute intense bronchitis, osteoarthritis, rheumatoid arthritis, infectious arthritis, post-infectious arthritis, gonocoele arthritis, tuberculous arthritis, arthritis, osteoarthritis, gout, spondyloarthropathies, ankylosing spondylitis, arthritis associated with vasculitis syndrome, nodular polyarteritis nervosa, irritable vasculitis, rugenic granulomatosis, rheumatoid polyposis myalgia, arthritis cell arteritis, calcium polycystic arthropathy, caustic gout, non-arthritic rheumatism, bursitis, hay fever, suppurative inflammation (e.g., tennis elbow), neuropathic joint disease, hemarthrosic, Henoch-Schlein purpura, hypertrophic osteoarthritis, multisized hemorrhoids, scoliosis, hemochromatosis, hyperlipoproteinemia, hypogammaglobulinemia, COPD, acute respiratory distress syndrome, acute lung injury, broncho-pulmonary dysplasia and systemic lupus erythematosus (SLE); and an epidermal disease or disorder, optionally wherein the epidermal disease or disorder is selected from the group consisting of psoriasis, atopic dermatitis, scleroderma, eczema, rosacea, seborrheic dermatitis, melanoma, solar keratosis, ichthyosis, Grover's disease, common warts, keratoacanthoma and seborrhoeic keratosis;
the nucleic acid-lipid particle, pharmaceutical composition, or injectate is administered parenterally, optionally wherein the nucleic acid-lipid particle, pharmaceutical composition, or injectate is administered via a route selected from the group consisting of inhalation, topical application and injection, optionally wherein the injection is selected from the group consisting of intravenous injection, intratracheal injection, intra-articular injection, subcutaneous injection, intradermal injection and intramuscular injection;
the nucleic acid cargo comprises a synthetic or naturally occurring RNA or DNA, or derivatives thereof, optionally wherein the nucleic acid cargo is a modified RNA, optionally wherein the modified RNA is selected from the group consisting of a modified mRNA, a modified antisense oligonucleotide and a modified siRNA, optionally wherein the modified mRNA encodes a nucleic acid modulating controller; and/or
the nucleic acid cargo comprises one or more modifications selected from the group consisting of 2′-O-methyl modified nucleotides, a nucleotide comprising a 5′-phosphorothioate group, a terminal nucleotide linked to a cholesteryl derivative, a 2′-deoxy-2′-fluoro modified nucleotide, a 5′-methoxy-modified nucleotide (e.g., 5′-methoxyuridine), a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide; internucleoside linkages or backbones including phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

59-63. (canceled)
